(19) United States
(12) Patent Application Publication
(10) Pub. No.: US 2017/0224764 A1
(43) Pub. Date: Aug. 10, 2017

(54) THERAPEUTIC TARGETING OF MITOCHONDRIA TO PREVENT OSTEOARTHRITIS

(71) Applicant: Cornell University, Ithaca, NY (US)

(72) Inventors: Michelle L. Delco, Ithaca, NY (US); Lisa Fortier, Ithaca, NY (US); Hazel H. Szeto, New York, NY (US)

(21) Appl. No.: 15/429,802

(22) Filed: Feb. 10, 2017

Related U.S. Application Data
(60) Provisional application No. 62/293,583, filed on Feb. 10, 2016.

(51) Int. Cl. A61K 38/12

(52) U.S. Cl. CPC A61K 38/12

(57) ABSTRACT

The present technology provides methods of preventing or treating osteoarthritis (OA) and/or post-traumatic osteoarthritis (PTOA). In some embodiments, the methods provide administering aromatic-cationic peptides in effective amounts to treat or prevent cartilage degeneration and/or chondrocyte death such as that found in a subject suffering from, or predisposed to OA or PTOA. In some embodiments, the methods comprise administering to a subject suffering from, or at risk for OA or PTOA, an effective amount of an aromatic-cationic peptide to subjects in need thereof.

### CROSS-REFERENCE TO RELATED APPLICATIONS

This application claims the benefit of and priority to U.S. Application No. 62/293,583 filed on Feb. 10, 2016, the content of which is incorporated by reference herein in its entirety.

### GOVERNMENT SUPPORT

The invention was made with U.S. Government support under grants T32RR007059 and 5T320D0011000-20 awarded by the National Institutes of Health. The U.S. Government has certain rights in the invention.

### TECHNICAL FIELD

The present technology relates generally to compositions and methods of preventing or treating osteoarthritis (OA) or post-traumatic osteoarthritis (PTOA). In particular, embodiments of the present technology relate to administering at least one aromatic-cationic peptide (e.g., D-Arg-2′,6′-dimethyltyrosine (Dmt)-Lys-Phe-NH₂; (SS-31)), or a pharmaceutically acceptable salt thereof, in effective amounts to treat or prevent cartilage degeneration in a subject suffering from OA or PTOA.

### BACKGROUND

The following description is provided to assist the understanding of the reader. None of the information provided or references cited is admitted to be prior art. Osteoarthritis (OA) is the leading cause of chronic disability, affecting over 67 million Americans. OA can generally be described as degenerative disease of articular cartilage; however, all joint tissues are affected including subchondral bone, synovium, and joint capsule. In most clinical cases, the etiopathogenesis of OA is multifactorial, with age, weight, disease, and genetics all likely playing a role. OA is also the number one cause of unsoundness in horses.

In addition to biological factors, OA is a disease of mechanics. Post-traumatic osteoarthritis (PTOA) develops secondary to joint trauma with clinical signs of pain and dysfunction often lagging years or decades behind the initiating injury. Trauma to cartilage can initiate PTOA. Approximately 12% of patients with symptomatic osteoarthritis (OA) had a traumatic incident to their joint as the inciting cause.

Talocrural (TC) joint (i.e., ankle), trauma is a common cause of OA. Unlike the knee and hip joints, where only 2-10% of OA is attributed to injury, up to 90% of arthritic change in the ankle is post-traumatic in nature. The ankle is the most commonly injured joint during sport activities, with >300,000 injuries per year reported in the U.S., and an estimated 52.3 ankle injuries per 1000 athletic exposures in high school-aged athletes. Ankle sprains are also the most common combat related injury, for example, ankle sprains have about a 15% incidence rate in over 4000 military personnel evaluated.

### SUMMARY OF THE PRESENT TECHNOLOGY

Generally, the present technology relates to the treatment, amelioration, or prevention of osteoarthritis (OA) through administration of a therapeutically effective amount of at least one aromatic-cationic peptide disclosed herein (e.g., D-Arg-2′,6′-Dmt-Lys-Phe-NH₂(SS-31)), or pharmaceutically acceptable salts thereof, such as acetate salt, tartrate salt, or trifluoroacetate salt, to a subject in need thereof.

In one aspect, the present technology relates to methods for treating or preventing osteoarthritis (OA) in a subject in need thereof comprising administering an effective amount D-Arg-2′,6′-Dmt-Lys-Phe-NH₂, or a pharmaceutically acceptable salt thereof. In some embodiments, the osteoarthritis is post-traumatic osteoarthritis (PTOA). In some embodiments, the OA is caused by mechanical injury. In some embodiments, the OA is located in the shoulder, hand, foot, ankle, toe, hip, spine, jaw, or knee. In some embodiments, the aromatic-cationic peptide, or pharmaceutically acceptable salt thereof, is administered orally, topically, intranasally, intraperitoneally, intravenously, subcutaneously, intraarticularly, or transdermally. In some embodiments, the peptide is administered within about 1 to 12 hours following mechanical injury. In some embodiments, the treatment or prevention comprises reducing or ameliorating one or more symptoms of osteoarthritis is selected from the group consisting of joint pain; joint swelling; joint clicking; joint cracking and/or creaking; joint stiffness; limited range of motion in a joint; pain in the groin, buttocks, inside knee, or thigh; grating or scraping sensation during movement of a knee; pain or tenderness in a toe joint; and swelling in ankles or toes.

In another aspect, the present technology relates to methods for treating or preventing post-traumatic osteoarthritis (PTOA) in a subject in need thereof comprising administering an effective amount D-Arg-2′,6′-Dmt-Lys-Phe-NH₂, or a pharmaceutically acceptable salt thereof. In some embodiments, the PTOA is caused by mechanical injury. In some embodiments, the PTOA is located in the shoulder, hand, foot, ankle, toe, hip, spine, jaw, or knee. In some embodiments, the D-Arg-2′,6′-Dmt-Lys-Phe-NH₂, or pharmaceutically acceptable salt thereof, is administered orally, topically, intranasally, intraperitoneally, intravenously, subcutaneously, intraarticularly, or transdermally. In some embodiments, the peptide is administered within about 1 to 12 hours following mechanical injury. In some embodiments, the treatment or prevention comprises reducing or ameliorating one or more symptoms of osteoarthritis is selected from the group consisting of joint pain; joint swelling; joint clicking; joint cracking and/or creaking; joint stiffness; limited range of motion in a joint; pain in the groin, buttocks, inside knee, or thigh; grating or scraping sensation during movement of a knee; pain or tenderness in a toe joint; and swelling in ankles or toes.

In another aspect, the present technology relates to methods for reducing cartilage degeneration and/or chondrocyte death after mechanical injury in a subject in need thereof comprising administering D-Arg-2′,6′-Dmt-Lys-Phe-NH₂, or a pharmaceutically acceptable salt thereof. In some embodiments, the cartilage degeneration and/or chondrocyte death is associated with osteoarthritis (OA) or post-traumatic osteoarthritis (PTOA). In some embodiments, the cartilage degeneration and/or chondrocyte death is caused by mechanical injury. In some embodiments, the cartilage degeneration and/or chondrocyte death is located in the shoulder, hand, foot, ankle, toe, hip, spine, jaw, or knee. In some embodiments, the aromatic-cationic peptide, or pharmaceutically acceptable salt thereof, is administered orally, topically, intranasally, intraperitoneally, intravenously, subcutaneously, intraarticularly, or transdermally. In some embodiments, the peptide is administered within about 1 to 6 hours following mechanical injury. In some embodiments, reducing cartilage degeneration and/or chondrocyte death reduces or ameliorates one or more symptoms of osteoarthritis is selected from the group consisting of joint pain; joint swelling; joint clicking; joint cracking and/or creaking; joint stiffness; limited range of motion in a joint; pain in the groin, buttocks, inside knee, or thigh; grating or scraping sensation during movement of a knee; pain or tenderness in a toe joint; and swelling in ankles or toes.

### BRIEF DESCRIPTION OF DRAWINGS

FIG. 1 is a diagram showing exemplary experimental design. The wells in the exemplary microrespirometry plate layout are as follows: Hi=high impact, Lo=low impact, C=no impact, L=left limb, R=right limb, BC=background [O₂] correction wells.

FIG. 2A is a graph showing respirometry in injured cartilage. Data represents OCR over time. Impacted cartilage from the PFG (both low impact and high impact) display lower basal OCRs (0-30 minutes) than controls and an altered response after addition of oligomycin (O), FCCCP (F), and rotenone/antimycin A (R+A). Background=[O₂] correction.

FIG. 2B is a graph showing respirometry in injured cartilage. Data represents OCR over time. The trend in FIG. 2A occurs in the condyle but basal OCRs are higher. Background=[O₂] correction.

FIGS. 3A-D are confocal images showing that cartilage injury results in mitochondria depolarization and cell death. FIGS. 3A and 3C are control cells and FIGS. 3B and 3D are impacted cells. Cells in FIGS. 3A and 3B are stained with Mitotracker green (green; all mitochondria, TMRM (red; only polarized mitochondria) and Hoechst 33342 (blue; nuclei). Green only stained mitochondria with increased uptake of nuclear stain indicates comprised integrity of both the cell and mitochondria membrane. FIGS. 3C and 3D are images of Calcein AM/ethidium homodimer (green/red; live/dead cell) staining of control (FIG. 3C) and impacted explants (FIG. 3D).

FIGS. 4A and 4B are images showing electron microscopy images of healthy mitochondria from uninjured cartilage (4A) and mitochondria from injured cartilage (4B) that show mitochondrial swelling and loss of membrane folds after injury.

FIGS. 5A-C are images showing that treatment with D-Arg-2′,6′-Dmt-Lys-Phe-NH₂ (SS-31) prevents impact-induced chondrocyte death. Control (5A), untreated, impacted cartilage (5B) and D-Arg-2′,6′-Dmt-Lys-Phe-NH₂, treated (treatment 1 hr after injury), impacted cartilage (5C) were stained for live (green) and dead (red) cells and imaged at 24 hrs.

FIG. 5D is a graph showing cell death in impacted cartilage treated at 0 or 1 hr is equivalent to un-injured controls.

FIG. 6 is a graph showing that treatment with D-Arg-2′,6′-Dmt-Lys-Phe-NH₂ (SS-31) prevents cell membrane damage after cartilage injury. Cartilage explants (n=18) were impacted then incubated for 7 days with or without D-Arg-2′,6′-Dmt-Lys-Phe-NH₂. LDH activity was measured in cartilage conditioned media every 24 hrs. Data is expressed as cumulative LDH activity over the culture period (*p<0.05).

FIGS. 7A and 7B are graphs showing that treatment with D-Arg-2′,6′-Dmt-Lys-Phe-NH₂ (SS-31) prevents cartilage matrix degradation after injury. Cartilage explants (n=18) were impacted then incubated with or without D-Arg-2′,6′-Dmt-Lys-Phe-NH₂ (SS-31). DMMB assay on cartilage conditioned media revealed GAG loss increased in non-treated, impacted samples by 96 hr post impact (*p<0.05) (FIG. 7A) and treatment with D-Arg-2′,6′-Dmt-Lys-Phe-NH₂ at 0, 1, or 6 hours after impact decreased GAG loss at 96 hr (*p<0.05) (FIG. 7B).

FIGS. 8A-D are images showing that treatment with D-Arg-2′,6′-Dmt-Lys-Phe-NH₂ (SS-31) prevents impact-induced cell death. Confocal images of healthy (8A, 8C) and impact-injured (8B, 8D) cartilage, fluorescently stained for live cells (green) and dead cells (red). The joint surface is toward the top. FIGS. 8A and 8B are untreated and FIGS. 8C and 8D were treated with D-Arg-2′,6′-Dmt-Lys-Phe-NH₂ (SS-31). There was a decrease in dead (red) cells after injury in D-Arg-2′,6′-Dmt-Lys-Phe-NH₂ treated cartilage (8D) versus untreated cartilage (8B).

FIG. 9A is an image showing an ex vivo cartilage impact device. Impactor mounted to armature, with talar OC block held in a vice grip, which rotates in 3-axis to allowing the impacting tip to be positioned perpendicular to articular surface.

FIG. 9B is an image showing that impact footprints were recorded using pressure sensitive film and surface area was measured using the ImageJ software masking function (inset).

FIG. 9C is an image showing that cartilage thickness was measured on cut section adjacent to impact sites using ImageJ software.

FIG. 9D are images showing live multiphoton images (10×) of control and impacted cartilage. Cartilage matrix cracks (arrows) and dead cells (circled) are present after impact.

FIG. 10A is an image showing the spring-loaded impacting device that was instrumented with an internal load cell to measure force and a linear variable displacement transducer (LVDT) to measure displacement. One of 2 impact tips were used (s and L; inset).

FIG. 10B is a graph showing the impact voltage signal from the load cell was converted to stress, using the contact area of each impact measured on pressure sensitive paper.

FIG. 10C is an exemplary image showing where multiple impacts were applied to each equine talus in areas (*; zones 4 and 6) corresponding to the highest incidence of osteochondral lesions in humans.

FIG. 11 is a graph showing the relationship between impact force and stress for the equine talus.

FIGS. 12A and 12B are images showing that a hand-held impactor creates cartilage lesions in vivo. FIG. 12A is an intraoperative arthroscopic view of 3 impacts (arrows) of varying magnitude created on the medial trochlea of the left talus in a horse subject. The impacting tip (star) is positioned through a standard arthroscopic portal within the joint. FIG. 12B is a post mortem dissection of the same joint. India ink was applied to the articular surface to mark impact sites (arrow heads).

FIGS. 13A-C are graphs showing that cartilage impact causes joint inflammation. FIG. 13A shows joint inflammation score, a combined measure of synovial fluid changes and clinical joint effusion remains elevated throughout the 12-week study. FIG. 13B shows PGE-2 concentrations were increased one week following impact, and returned to baseline levels within 4 weeks. Error bars=±s.d. FIG. 13C shows synovial histopathology was scored for inflammation, vascularity and subintimal edema, and revealed mild to moderate synovitis at 6 weeks (joints 3 and 4) and 12 weeks (joints 1 and 2) post-impact. Control data represent the average of 2 joints.

FIG. 13D is an image that represents 20× images of synovial sections stained with hematoxylin and eosin from control and injured joints.

FIGS. 14A and 14B are graphs showing that impact causes early OA-like osteochondral lesions and cartilage damage was correlated with impact stress. FIG. 14A shows that an average OARSI score by experimental joint (1-4) revealed moderate to severe OA at the impact sites (grey) and mild changes in the two non-impacted areas within the experimental joints (black). Error bars=±s.d. FIG. 14B shows an OARSI score at 12 weeks correlated with peak impact stress.

FIG. 14C is an image that represents 10× images of osteochondral sections stained with safranin O/fast green (SOFG) and hematoxylin and eosin (H&E) of OARSI grades 0, 3, 4 and 5. Note the persistence of India ink (black) applied at necropsy, marking impacts. Bars=150 μm.

FIG. 15 is an image showing an exemplary experimental design and methods. By way of example, but not by way of limitation, cartilage explants were harvested from the medial femoral condyle (MFC) for the first set of experiments, and from 2 sites for the second set of experiments; the MFC and the distal patellofemoral groove (PFG). One explant from each region was impacted at a higher impact magnitude, one was impacted at a lower magnitude, and one served as an unimpacted control. Explants were then divided for use in several assays; chondrocyte viability was quantified using live/dead staining, mitochondrial membrane polarity was determined as red to green fluorescent intensity (R:G) ratio on confocal imaging and mitochondrial respiratory function was assessed via microrespirometry. Cell membrane damage was assessed by measuring lactate dehydrogenase (LDH) activity in cartilage conditioned media.

FIG. 16 is a graph showing that chondrocyte death is correlated with impact magnitude. Cell death was positively correlated with peak impact stress for both the MFC and PFG (PFG r²=0.79, p<0.001; MFC r²=0.70, p<0.0001).

FIGS. 17A-C are graphs showing that respirometry assays indicate that acute impact induces mitochondrial dysfunction in cartilage. Cartilage from the medial femoral condyle was impacted at various magnitudes (M1-M4) and mitochondrial respiration was quantified by measuring oxygen consumption rate (OCR), then normalizing data to live cell number for each explant. FIG. 17A shows curves for OCR versus time for control, low impact (M2), and high impact (M4) groups. The graph demonstrates the differences in mitochondrial respiratory function between groups. Note that oligomycin-inhibited respiration does not reach steady state (121 minutes), but Rot+AA inhibited respiration does (225 minutes.) FIG. 17B shows B\baseline OCR (bOCR). FIG. 17C maximum respiration (mOCR) decreased with increasing impact magnitude (M1-M4). Groups that do not share a letter are significantly different at p<0.05. Error bars=±s.d.

FIGS. 18A and 18B are graphs showing cartilage impact results in cell membrane damage. Cell membrane damage was quantified in cartilage explants from the medial femoral condyle by performing lactate dehydrogenase (LDH) activity assay on cartilage-conditioned media following respirometry assay. FIG. 18A shows that cell damage was increased in explants impacted at higher magnitudes (M3 and M4) compared to un-impacted controls. Asterisks denote significant increase compared to controls at p<0.05. FIG. 18B shows that LDH activity in cartilage conditioned media peaks at approximately 5-7 hours after impact. Error bars=±s.d.

FIGS. 19A-C show images of PFG cartilage stained for live cells (green) with calcein AM and dead cells (red) with ethidium homodimer and imaged in cross-section using confocal microscopy. FIG. 19A shows un-impacted control cartilage had less dead (red) staining than FIG. 19B, which show lower impacted (M1) PFG explants and FIG. 19C, which shows higher impacted (M2) PFG explants.

FIG. 19D is a graph showing that impact-induced chondrocyte death differs by location within the joint. Chondrocytes from the patellofemoral groove (PFG) were more sensitive to impact-induced cell death than the medial femoral condyle (MFC). At lower impact magnitudes (M1) MFC viability was not affected. Groups that do not share a letter are significantly different at p<0.05. Error bars=±s.d.

FIG. 20 is a graph showing that the patellofemoral groove (PFG) is more sensitive to impact-induced mitochondrial respiratory dysfunction than the medial femoral condyle (MFC). The basal oxygen consumption rate (bOCR) of viable chondrocytes was significantly lower in PFG cartilage (red box and whisker plots) impacted at the lowest (M1) and higher (M2) magnitudes compared to un-injured control cartilage, whereas in MFC cartilage (blue box and whisker plots), bOCR is only affected at the higher impact magnitude (M2). Groups that do not share a letter are significantly different at p<0.05. Error bars=±s.d.

FIG. 21A is an image showing that the patellofemoral groove (PFG) is more sensitive to impact-induced mitochondria depolarization than the medial femoral condyle (MFC). FIG. 21A shows confocal images of control and impacted (M2) PFG explants stained for mitochondrial polarity at low (top) and high (bottom) magnification. Cartilage is stained with Mitotracker Green (green; all mitochondria), tetramethylrhodamine methyl ester perchlorate (red; polarized/functional mitochondria), and Hoechst 33342 (blue; nuclear counterstain, higher affinity for cells with compromised cell membranes). Red:green fluorescent intensity ratios were calculated on an image-wide basis using multiple low magnification z-stacks for each explant (top) using a custom ImageJ macro. This technique was validated on a single-cell basis by manually drawing ROIs around single cells at higher magnification (bottom).

FIG. 21B is a graph showing that mitochondria depolarization occurred in PFG cartilage from both the lower (M1) and higher impact (M2) groups compared to PFG controls. Significant differences were not detected between impact groups from the MFC. Asterisks denote a significant difference compared to control at p<0.05. Error bars=±s.d.

FIG. 22 is an image of an exemplary experimental design. Half the cartilage explants were impacted (X) at time 0. Injured groups (I; red bars) and non-injured groups (C; grey bars) were then treated (O) with D-Arg-2′,6′-Dmt-Lys-Phe-NH₂ (SS-31) 1 μM at time zero (T₀), 1 hour after injury (T₁), 6 hr after injury (T₆), 12 hr after injury (T₁₂) or left untreated (Tₙₒ). Explants were imaged on day 1 or 7 for cell death or apoptosis, and cartilage conditioned medium was collected at 1, 6, and 12 hours, and 1, 3, 5, and 7 days after injury to assess cartilage matrix degeneration (GAG loss) and cell membrane damage.

FIGS. 23A and 23B are graphs showing that treatment with D-Arg-2′,6′-Dmt-Lys-Phe-NH₂ (SS-31) prevents chondrocyte death. FIG. 23A shows that chondrocyte death (% dead cells) in injured explants treated with D-Arg-2′,6′-Dmt-Lys-Phe-NH₂ (SS-31) at 0, 1, or 6 hours was equivalent to uninjured controls. Timing of treatment did not affect chondrocyte viability. FIG. 23B shows that D-Arg-2′,6′-Dmt-Lys-Phe-NH₂ (SS-31) was effective at preventing chondrocyte death on day 1 and 7 post-impact.

FIG. 23C shows confocal images of uninjured (control), injured (impact) and injured, treated (impact+SS-31) cartilage on day 1 and 7. Explants were stained for live and dead cells with calcein AM (green) and ethidium homodimer (red), respectively. Groups that do not share a letter are significantly different at p≦0.05. Error bars=±s.d.

FIG. 24A is a graph showing that treatment with D-Arg-2′,6′-Dmt-Lys-Phe-NH₂ (SS-31) prevents apoptosis. Apoptosis in injured explants treated with D-Arg-2′,6′-Dmt-Lys-Phe-NH₂ (SS-31) was equivalent to uninjured controls at day 1 and 7.

FIG. 24B are confocal images of uninjured (control), injured (impact) and injured, treated (impact+SS-31) cartilage on day 1 and 7. Explants were stained for activated caspase 3 and 7 (caspase+) and imaged using reflectance to highlight collagen in the extracellular matrix (matrix). Groups that do not share a letter are significantly different at p<0.05. Error bars=±s.d.

FIGS. 25A and 25B are graphs showing that treatment with D-Arg-2′,6′-Dmt-Lys-Phe-NH₂ (SS-31) prevents chondrocyte membrane damage and cartilage matrix degradation. FIG. 25A shows LDH activity in the media of injured, D-Arg-2′,6′-Dmt-Lys-Phe-NH₂ treated groups is lower than injured, untreated explants, and similar to uninjured controls. D-Arg-2′,6′-Dmt-Lys-Phe-NH₂ (SS-31) also has a protective effect against cell membrane damage in treated controls (p=0.05). FIG. 25B shows that cumulative GAG loss into the media on days 3-7 was increased in injured, untreated explants compared to uninjured controls. GAG loss was similar in injured, treated and control groups. Groups that do not share a letter are significantly different at p<0.05. Error bars=±s.d.

FIG. 26A is a graph showing that chondrocyte death (% dead cells) in injured explants (n=6/group) treated with D-Arg-2′,6′-Dmt-Lys-Phe-NH₂ (SS-31) at 0, 1, 6, or 12 hours was reduced as compared to untreated, injured controls. Groups that do not share a letter are significantly different at p<0.05. Error bars=±s.d.

FIG. 26B is a graph showing that cartilage matrix degeneration, measured by glycosaminoglycan (GAG) loss into the media, in explants treated with D-Arg-2′,6′-Dmt-Lys-Phe-NH₂ (SS-31) at 0, 1, or 6 hours was equivalent to uninjured controls and that explants treated with D-Arg-2′,6′-Dmt-Lys-Phe-NH₂ at 12 hours had reduced cartilage matrix degeneration as compared to untreated, injured controls. Groups that do not share a letter are significantly different at p<0.05. Error bars=±s.d.

### DETAILED DESCRIPTION

It is to be appreciated that certain aspects, modes, embodiments, variations and features of the present technology are described below in various levels of detail in order to provide a substantial understanding of the present technology. The present technology provides methods comprising administering at least one aromatic-cationic peptide, e.g., D-Arg-2′,6′-Dmt-Lys-Phe-NH₂, or a pharmaceutically acceptable salt thereof, in effective amounts to treat, prevent, or ameliorate OA or PTOA in a subject in need thereof.

The present disclosure contemplates neutral (non-salt) aromatic-cationic peptides disclosed herein, all salts of the peptides, and methods of using neutral and salt forms of the peptides. In some embodiments, the salts of the aromatic-cationic peptides comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). A salt of a basic peptide may be prepared by methods known in the art, such as by treating the peptide or a composition comprising the peptide with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. A salt of an acidic peptide can be prepared by methods known in the art, such as by treating the peptide or a composition comprising the peptide with a base. Examples of inorganic salts of acidic peptides include, but are not limited to, alkali metal and alkaline earth salts, such as sodium salts, potassium salts, magnesium salts, and calcium salts; ammonium salts; and aluminum salts.

Examples of organic salts of acidic peptides include, but are not limited to, procaine, dibenzylamine, N-ethylpiperidine, N,N′-dibenzylethylenediamine, and triethylamine salts. The present technology also includes all stereoisomers and geometric isomers of the peptides, including diastereomers, enantiomers, and cis/trans (E/Z) isomers. The present technology also includes mixtures of stereoisomers and/or geometric isomers in any ratio, including, but not limited to, racemic mixtures.

### Definitions

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include plural referents unless the content clearly dictates otherwise. For example, reference to “a cell” includes a combination of two or more cells, and the like.

As used herein, the “administration” of an agent, drug, or peptide to a subject includes any route of introducing or delivering to a subject a compound to perform its intended function. Administration can be carried out by any suitable route, including orally, intranasally, parenterally (intravenously, intramuscularly, intraperitoneally, or subcutaneously), intraarticularly, or topically. In some embodiments, the aromatic-cationic peptide is administered by an intracoronary route or an intra-arterial route. Administration includes self-administration and the administration by another.

As used herein, the term “effective amount” refers to a quantity of a composition sufficient to achieve a desired therapeutic and/or prophylactic effect, e.g., an amount which results in the treatment of prevention of OA or PTOA and/or one or more symptoms associated with OA or PTOA. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan would be able to determine appropriate dosages depending on these and other factors. Compositions of the present technology can be administered alone or in combination with one or more additional therapeutic compounds, such as compounds know in the art for treating or preventing OA or PTOA. In some embodiments peptides or compositions of the present technology are administered to a subject having one or more signs or symptoms of OA or PTOA. For example, a “therapeutically effective amount” of the aromatic-cationic peptides means levels in which the physiological effects of OA or PTOA are, at a minimum, ameliorated. A therapeutically effective amount can be given in one or more administrations. In some embodiments, signs, symptoms or complications of OA or PTOA include, but are not limited to, joint pain, swelling of joint, clicking, cracking, and/or creaking of joints, stiff joints, limited range of motion in joint, pain in groin, buttocks, or inside knee or thigh, grating or scraping sensation during movement of knee, pain and tenderness in large joint at base of big toe, and swelling in ankles or toes.

As used herein, “mechanical injury” refers to a physical force to the body or at least one part of the body that overloads and destabilizes at least one joint and results in tissue damage within the joint. By way of example, but not by way of limitation, in some embodiments, physical force is one or more forces selected from the group consisting of an insult, a blow, an impact, compression, twisting, over use, or pulling. By way of example, but not by way of limitation, in some embodiments, the part of the body is the shoulder, hand, foot, ankle, toe, hip, spine, jaw, or knee.

As used herein, “prevention” or “preventing” of a disorder or condition refers to a compound that, in a statistical sample, reduces the occurrence of the disorder or condition in the sample relative to a control sample, or delays the onset or reduces the severity of one or more symptoms of the disorder or condition relative to the control sample. As used herein, preventing OA or PTOA includes preventing chondrocyte death and cartilage degeneration.

As used herein, the terms “subject,” “individual,” or “patient” can be an individual organism, such as a vertebrate, a mammal, or a human.

As used herein, the terms “treating” or “treatment” or “alleviation” refer to therapeutic treatment, wherein the object is to prevent or slow down (lessen) the targeted pathologic condition or disorder. A subject is successfully “treated” for OA or PTOA if, after receiving a therapeutic amount of the aromatic-cationic peptides according to the methods described herein, the subject shows observable and/or measurable reduction in cartilage degeneration and/or chondrocyte death. It is also to be appreciated that the various modes of treatment or prevention of medical conditions as described are intended to mean “substantial,” which includes total but also less than total treatment or prevention, and wherein some biologically or medically relevant result is achieved.

### Osteoarthritis

Osteoarthritis is a form of arthritis that features the breakdown and eventual loss of the cartilage in one or more joints. OAs can affect the hands, feet, ankle, spine, and large weight-hearing joints, such as the hips and knees.

OA that develops secondary to a wide variety of joint injury is often grouped into a sub-catergory of OA called post-traumatic osteoarthritis (PTOA). Common injuries that can lead to PTOA include, but are not limited to, high-speed impact trauma to the articular surface, intraarticular fractures, and joint-destabilizing soft-tissue tears. Although the end-stage pathophysiology of PTOA may be similar, there is evidence to suggest that the early biological and mechanical events that initiate and perpetuate disease are distinct between different joints, injury types, and patient populations. The ankle, knee, and hip are the most commonly injured joints in PTOA.

The most common injury precipitating end-stage ankle OA is a severe ankle sprain, when rapid ankle inversion causes the distal tibia to impact the medial aspect of the talar dome, often resulting in an osteochondral lesion. Ligamentous injuries commonly accompany severe ankle sprains and may result in joint instability. The magnitude of the initial cartilage trauma is an important factor in development of ankle PTOA.

The present technology relates to treating, preventing, or ameliorating OA or PTOA in a subject in need thereof, by administering at least one aromatic-cationic peptide as disclosed herein, such as D-Arg-2′,6′-Dmt-Lys-Phe-NH₂, or pharmaceutically acceptable salts thereof, such as acetate salt, tartrate salt, or trifluoroacetate salt. The present technology relates to the treatment, amelioration, or prevention of OA or PTOA in mammals through administration of therapeutically effective amounts of aromatic-cationic peptides as disclosed herein, such as D-Arg-2′,6′-Dmt-Lys-Phe-NH₂, or pharmaceutically acceptable salts thereof, such as acetate salt, tartrate salt, or trifluoroacetate salt, to subjects in need thereof.

### Aromatic-Cationic Peptides of the Present Technology

The aromatic-cationic peptides are water-soluble and highly polar. Despite these properties, the peptides can readily penetrate cell membranes. The aromatic-cationic peptides typically include a minimum of three amino acids or a minimum of four amino acids, covalently joined by peptide bonds. The maximum number of amino acids present in the aromatic-cationic peptides is about twenty amino acids covalently joined by peptide bonds. Suitably, the maximum number of amino acids is about twelve, about nine, or about six.

The amino acids of the aromatic-cationic peptides can be any amino acid. As used herein, the term “amino acid” is used to refer to any organic molecule that contains at least one amino group and at least one carboxyl group. Typically, at least one amino group is at the a position relative to a carboxyl group. The amino acids may be naturally occurring. Naturally occurring amino acids include, for example, the twenty most common levorotatory (L) amino acids normally found in mammalian proteins, i.e., alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val). Other naturally occurring amino acids include, for example, amino acids that are synthesized in metabolic processes not associated with protein synthesis. For example, the amino acids ornithine and citrulline are synthesized in mammalian metabolism during the production of urea. Another example of a naturally occurring amino acid includes hydroxyproline (Hyp).

The peptides optionally contain one or more non-naturally occurring amino acids. Optimally, the peptide has no amino acids that are naturally occurring. The non-naturally occurring amino acids may be levorotary (L-), dextrorotatory (D-), or mixtures thereof. Non-naturally occurring amino acids are those amino acids that typically are not synthesized in normal metabolic processes in living organisms, and do not naturally occur in proteins. In addition, the non-naturally occurring amino acids suitably are also not recognized by common proteases. The non-naturally occurring amino acid can be present at any position in the peptide. For example, the non-naturally occurring amino acid can be at the N-terminus, the C-terminus, or at any position between the N-terminus and the C-terminus.

The non-natural amino acids may, for example, comprise alkyl, aryl, or alkylaryl groups not found in natural amino acids. Some examples of non-natural alkyl amino acids include α-aminobutyric acid, β-aminobutyric acid, γ-aminobutyric acid, δ-aminovaleric acid, and ε-aminocaproic acid. Some examples of non-natural aryl amino acids include ortho-, meta, and para-aminobenzoic acid. Some examples of non-natural alkylaryl amino acids include ortho-, meta-, and para-aminophenylacetic acid, and γ-phenyl-β-aminobutyric acid. Non-naturally occurring amino acids include derivatives of naturally occurring amino acids. The derivatives of naturally occurring amino acids may, for example, include the addition of one or more chemical groups to the naturally occurring amino acid.

For example, one or more chemical groups can be added to one or more of the 2′, 3′, 4′,5′, or 6′ position of the aromatic ring of a phenylalanine or tyrosine residue, or the 4′, 5′,6′, or 7′ position of the benzo ring of a tryptophan residue. The group can be any chemical group that can be added to an aromatic ring. Some examples of such groups include branched or unbranched C₁-C₄ alkyl, such as methyl, ethyl, n-propyl, isopropyl, butyl, isobutyl, or t-butyl, C₁-C₄ alkyloxy (i.e., alkoxy), amino, C₁-C₄ alkylamino and C₁-C₄ dialkylamino (e.g., methylamino, dimethylamino), nitro, hydroxyl, halo (i.e., fluoro, chloro, bromo, or iodo). Some specific examples of non-naturally occurring derivatives of naturally occurring amino acids include norvaline (Nva) and norleucine (Nle).

Another example of a modification of an amino acid in a peptide is the derivatization of a carboxyl group of an aspartic acid or a glutamic acid residue of the peptide. One example of derivatization is amidation with ammonia or with a primary or secondary amine, e.g., methylamine, ethylamine, dimethylamine or diethylamine. Another example of derivatization includes esterification with, for example, methyl or ethyl alcohol. Another such modification includes derivatization of an amino group of a lysine, arginine, or histidine residue. For example, such amino groups can be acylated. Some suitable acyl groups include, for example, a benzoyl group or an alkanoyl group comprising any of the C₁-C₄ alkyl groups mentioned above, such as an acetyl or propionyl group.

The non-naturally occurring amino acids are suitably resistant or insensitive, to common proteases. Examples of non-naturally occurring amino acids that are resistant or insensitive to proteases include the dextrorotatory (D-) form of any of the above-mentioned naturally occurring L-amino acids, as well as L- and/or D- non-naturally occurring amino acids. The D-amino acids do not normally occur in proteins, although they are found in certain peptide antibiotics that are synthesized by means other than the normal ribosomal protein synthetic machinery of the cell. As used herein, the D-amino acids are considered to be non-naturally occurring amino acids.

In order to minimize protease sensitivity, the peptides should have less than five, less than four, less than three, or less than two contiguous L-amino acids recognized by common proteases, irrespective of whether the amino acids are naturally or non-naturally occurring. In one embodiment, the peptide has only D-amino acids, and no L-amino acids. If the peptide contains protease sensitive sequences of amino acids, at least one of the amino acids is preferably a non-naturally-occurring D-amino acid, thereby conferring protease resistance. An example of a protease sensitive sequence includes two or more contiguous basic amino acids that are readily cleaved by common proteases, such as endopeptidases and trypsin. Examples of basic amino acids include arginine, lysine and histidine.

The aromatic-cationic peptides should have a minimum number of net positive charges at physiological pH in comparison to the total number of amino acid residues in the peptide. The minimum number of net positive charges at physiological pH will be referred to below as (pₘ). The total number of amino acid residues in the peptide will be referred to below as (r). The minimum number of net positive charges discussed below is all at physiological pH. The term “physiological pH” as used herein refers to the normal pH in the cells of the tissues and organs of the mammalian body. For instance, the physiological pH of a human is normally approximately 7.4, but normal physiological pH in mammals may be any pH from about 7.0 to about 7.8.

“Net charge” as used herein refers to the balance of the number of positive charges and the number of negative charges carried by the amino acids present in the peptide. In this specification, it is understood that net charges are measured at physiological pH. The naturally occurring amino acids that are positively charged at physiological pH include L-lysine, L-arginine, and L-histidine. The naturally occurring amino acids that are negatively charged at physiological pH include L-aspartic acid and L-glutamic acid.

Typically, a peptide has a positively charged N-terminal amino group and a negatively charged C-terminal carboxyl group. The charges cancel each other out at physiological pH. As an example of calculating net charge, the peptide Tyr-Arg-Phe-Lys-Glu-His-Trp-D-Arg has one negatively charged amino acid (i.e., Glu) and four positively charged amino acids (i.e., two Arg residues, one Lys, and one His). Therefore, the above peptide has a net positive charge of three.

In one embodiment, the aromatic-cationic peptides have a relationship between the minimum number of net positive charges at physiological pH (pₘ) and the total number of amino acid residues (r) wherein 3pₘ is the largest number that is less than or equal to r+1. In this embodiment, the relationship between the minimum number of net positive charges (pₘ) and the total number of amino acid residues (r) is as follows:

TABLE 1
Amino acid number and net positive charges (3pₘ ≦ p + 1)

| (r) | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|
| (pₘ) | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of net positive charges (pₘ) and the total number of amino acid residues (r) wherein 2pₘ is the largest number that is less than or equal to r+1. In this embodiment, the relationship between the minimum number of net positive charges (pₘ) and the total number of amino acid residues (r) is as follows:

TABLE 2
Amino acid number and net positive charges (2pₘ ≦ p + 1)

| (r) | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|
| (pₘ) | 2 | 2 | 3 | 3 | 4 | 4 | 5 | 5 | 6 | 6 | 7 | 7 | 8 | 8 | 9 | 9 | 10 | 10 |

In one embodiment, the minimum number of net positive charges (pₘ) and the total number of amino acid residues (r) are equal. In another embodiment, the peptides have three or four amino acid residues and a minimum of one net positive charge, suitably, a minimum of two net positive charges and more preferably a minimum of three net positive charges.

It is also important that the aromatic-cationic peptides have a minimum number of aromatic groups in comparison to the total number of net positive charges (pₜ). The minimum number of aromatic groups will be referred to below as (a). Naturally occurring amino acids that have an aromatic group include the amino acids histidine, tryptophan, tyrosine, and phenylalanine. For example, the hexapeptide Lys-Gln-Tyr-D-Arg-Phe-Trp has a net positive charge of two (contributed by the lysine and arginine residues) and three aromatic groups (contributed by tyrosine, phenylalanine and tryptophan residues).

The aromatic-cationic peptides should also have a relationship between the minimum number of aromatic groups (a) and the total number of net positive charges at physiological pH (pₜ) wherein 3a is the largest number that is less than or equal to pₜ+1, except that when pₜ is 1, a may also be 1. In this embodiment, the relationship between the minimum number of aromatic groups (a) and the total number of net positive charges (pₜ) is as follows:

TABLE 3
Aromatic groups and net positive charges (3a ≦ pₜ + 1 or a = pₜ = 1)

| (pₜ) | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| (a) | 1 | 1 | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of aromatic groups (a) and the total number of net positive charges (pₜ) wherein 2a is the largest number that is less than or equal to pₜ+1. In this embodiment, the relationship between the minimum number of aromatic amino acid residues (a) and the total number of net positive charges (pₜ) is as follows:

TABLE 4
Aromatic groups and net positive charges (2a ≦ pₜ + 1 or a = pₜ = 1)

| (pₜ) | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| (a) | 1 | 1 | 2 | 2 | 3 | 3 | 4 | 4 | 5 | 5 | 6 | 6 | 7 | 7 | 8 | 8 | 9 | 9 | 10 | 10 |

In another embodiment, the number of aromatic groups (a) and the total number of net positive charges (pₜ) are equal.

Carboxyl groups, especially the terminal carboxyl group of a C-terminal amino acid, are suitably amidated with, for example, ammonia to form the C-terminal amide. Alternatively, the terminal carboxyl group of the C-terminal amino acid may be amidated with any primary or secondary amine. The primary or secondary amine may, for example, be an alkyl, especially a branched or unbranched C₁-C₄ alkyl, or an aryl amine.

Accordingly, the amino acid at the C-terminus of the peptide may be converted to an amido, N-methylamido, N-ethylamido, N,N-dimethylamido, N,N-diethylamido, N-methyl-N-ethylamido, N-phenylamido or N-phenyl-N-ethylamido group. The free carboxylate groups of the asparagine, glutamine, aspartic acid, and glutamic acid residues not occurring at the C-terminus of the aromatic-cationic peptides may also be amidated wherever they occur within the peptide. The amidation at these internal positions may be with ammonia or any of the primary or secondary amines described above.

In one embodiment, the aromatic-cationic peptide is a tripeptide having two net positive charges and at least one aromatic amino acid. In a particular embodiment, the aromatic-cationic peptide is a tripeptide having two net positive charges and two aromatic amino acids.

In yet another aspect, the present technology provides an aromatic-cationic peptide or a pharmaceutically acceptable salt thereof such as acetate, tartrate, or trifluoroacetate salt. In some embodiments, the peptide comprises
1. at least one net positive charge;
2. a minimum of three amino acids;
3. a maximum of about twenty amino acids;
4. a relationship between the minimum number of net positive charges (pₘ) and the total number of amino acid residues (r) wherein 3pₘ is the largest number that is less than or equal to r+1; and
5. a relationship between the minimum number of aromatic groups (a) and the total number of net positive charges (pₜ) wherein 2a is the largest number that is less than or equal to pₜ+1, except that when a is 1, pₜ may also be 1.

In some embodiments, the peptide comprises the amino acid sequence Tyr-D-Arg-Phe-Lys-NH₂ (SS-01), 2′,6′-Dmt-D-Arg-Phe-Lys-NH₂ (SS-02), Phe-D-Arg-Phe-Lys-NH₂ (SS-20) or D-Arg-2′,6′-Dmt-Lys-Phe-NH₂ (SS-31). In some embodiments, the peptide comprises one or more of:
D-Arg-Dmt-Lys-Trp-NH₂;
D-Arg-Trp-Lys-Trp-NH₂;
D-Arg-2′,6′-Dmt-Lys-Phe-Met-NH₂;
H-D-Arg-Dmt-Lys(N^{a}Me)-Phe-NH₂;
H-D-Arg-Dmt-Lys-Phe(NMe)-NH₂;
H-D-Arg-Dmt-Lys(N^{α}Me)-Phe(NMe)-NH₂;
H-D-Arg(N^{α}Me)-Dmt(NMe)-Lys(N^{α}Me)-Phe(NMe)NH₂;
D-Arg-Dmt-Lys-Phe-Lys-Trp-NH₂;
D-Arg-Dmt-Lys-Dmt-Lys-Trp-NH₂;
D-Arg-Dmt-Lys-Phe-Lys-Met-NH₂;
D-Arg-Dmt-Lys-Dmt-Lys-Met-NH₂;
H-D-Arg-Dmt-Lys-Phe-Sar-Gly-Cys-NH₂;
H-D-Arg-Ψ[CH₂—NH]Dmt-Lys-Phe-NH₂;
H-D-Arg-Dmt-Ψ[CH₂—NH]Lys-Phe-NH₂;
H-D-Arg-Dmt-LysΨ[CH₂—NH]Phe-NH₂;
H-D-Arg-Dmt-Ψ[CH₂—NH]Lys-Ψ[CH₂—NH]Phe-NH₂;
Lys-D-Arg-Tyr-NH₂;
Tyr-D-Arg-Phe-Lys-NH₂;
2′,6′-Dmt-D-Arg-Phe-Lys-NH₂;
Phe-D-Arg-Phe-Lys-NH₂;
Phe-D-Arg-Dmt-Lys-NH₂;
D-Arg-2′6′Dmt-Lys-Phe-NH₂;
H-Phe-D-Arg-Phe-Lys-Cys-NH₂;
Lys-D-Arg-Tyr-NH₂;
D-Tyr-Trp-Lys-NH₂;
Trp-D-Lys-Tyr-Arg-NH₂;
Tyr-His-D-Gly-Met;
Tyr-D-Arg-Phe-Lys-Glu-NH₂;
Met-Tyr-D-Lys-Phe-Arg;
D-His-Glu-Lys-Tyr-D-Phe-Arg;
Lys-D-Gln-Tyr-Arg-D-Phe-Trp-NH₂;
Phe-D-Arg-Lys-Trp-Tyr-D-Arg-His;
Gly-D-Phe-Lys-Tyr-His-D-Arg-Tyr-NH₂;
Val-D-Lys-His-Tyr-D-Phe-Ser-Tyr-Arg-NH₂;
Trp-Lys-Phe-D-Asp-Arg-Tyr-D-His-Lys;
Lys-Trp-D-Tyr-Arg-Asn-Phe-Tyr-D-His-NH₂;
Thr-Gly-Tyr-Arg-D-His-Phe-Trp-D-His-Lys;
Asp-D-Trp-Lys-Tyr-D-His-Phe-Arg-D-Gly-Lys-NH₂;
D-His-Lys-Tyr-D-Phe-Glu-D-Asp-D-His-D-Lys-Arg-Trp-NH₂;
Ala-D-Phe-D-Arg-Tyr-Lys-D-Trp-His-D-Tyr-Gly-Phe;
Tyr-D-His-Phe-D-Arg-Asp-Lys-D-Arg-His-Trp-D-His-Phe;
Phe-Phe-D-Tyr-Arg-Glu-Asp-D-Lys-Arg-D-Arg-His-Phe-NH₂;
Phe-Tyr-Lys-D-Arg-Trp-His-D-Lys-D-Lys-Glu-Arg-D-Tyr-Thr;
Tyr-Asp-D-Lys-Tyr-Phe-D-Lys-D-Arg-Phe-Pro-D-Tyr-His-Lys;
Glu-Arg-D-Lys-Tyr-D-Val-Phe-D-His-Trp-Arg-D-Gly-Tyr-Arg-D-Met-NH₂;
Arg-D-Leu-D-Tyr-Phe-Lys-Glu-D-Lys-Arg-D-Trp-Lys-D-Phe-Tyr-D-Arg-Gly;
D-Glu-Asp-Lys-D-Arg-D-His-Phe-Phe-D-Val-Tyr-Arg-Tyr-D-Tyr-Arg-His-Phe-NH₂;
Asp-Arg-D-Phe-Cys-Phe-D-Arg-D-Lys-Tyr-Arg-D-Tyr-Trp-D-His-Tyr-D-Phe-Lys-Phe;
His-Tyr-D-Arg-Trp-Lys-Phe-D-Asp-Ala-Arg-Cys-D-Tyr-His-Phe-D-Lys-Tyr-His-Ser-NH₂;
Gly-Ala-Lys-Phe-D-Lys-Glu-Arg-Tyr-His-D-Arg-D-Arg-Asp-Tyr-Trp-D-His-Trp-His-D-Lys-Asp;
Thr-Tyr-Arg-D-Lys-Trp-Tyr-Glu-Asp-D-Lys-D-Arg-His-Phe-D-Tyr-Gly-Val-Ile-D-His-Arg-Tyr-Lys-NH₂;
Dmt-D-Arg-Phe-(atn)Dap-NH₂, where (atn)Dap is β-anthraniloyl-L-α,β-diaminopropionic acid;
Dmt-D-Arg-Ald-Lys-NH₂, where Ald is β-(6′-dimethylamino-2′-naphthoyl)alanine;
Dmt-D-Arg-Phe-Lys-Aid-NH₂, where Ald is β-(6′-dimethylamino-2′-naphthoyl)alanine
Dmt-D-Arg-Phe-(dns)Dap-NH₂ where (dns)Dap is β-dansyl-L-α,β-diaminopropionic acid;
D-Arg-Tyr-Lys-Phe-NH₂; and
D-Arg-Tyr-Lys-Phe-NH₂.

In some embodiments, “Dmt” refers to 2′,6′-dimethyltyrosine (2′,6′-Dmt) or 3′,5′-dimethyltyrosine (3′5′Dmt).

In some embodiments, the peptide is defined by formula I:

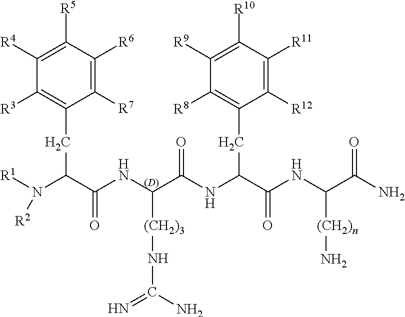

wherein R¹ and R² are each independently selected from

(i) hydrogen;

(ii) linear or branched C₁-C₆ alkyl;

(iii)

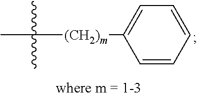

(iv)

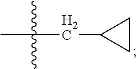 [SMILES: *CC1CC1]

(v)

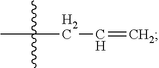 [SMILES: *CC=C]

R³, R⁴, R⁵, R⁶, R⁷, R⁸, R⁹, R¹⁰, R¹¹ and R¹² are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo; and n is an integer from 1 to 5.

In some embodiments, R¹, R², R³, R⁴, R⁵, R⁶, R⁷, R⁸, R⁹, R¹⁰, R¹¹ and R¹² are all hydrogen; and n is 4. In another embodiment, R¹, R², R³, R⁴, R⁵, R⁶, R⁷, R⁸, R⁹, and R¹¹ are all hydrogen; R⁸ and R¹² are methyl; R¹⁰ is hydroxyl; and n is 4.

In some embodiments, the peptide is defined by formula II:

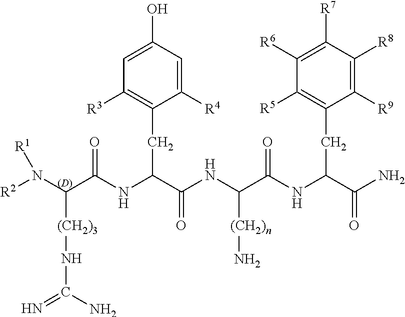

wherein R¹ and R² are each independently selected from

(i) hydrogen;

(ii) linear or branched C₁-C₆ alkyl;

(iii)

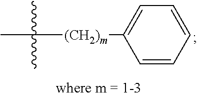

(iv)

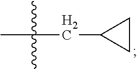 [SMILES: *CC1CC1]

(v)

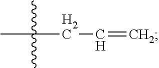 [SMILES: *CC=C]

R³ and R⁴ are each independently selected from

(i) hydrogen;

(ii) linear or branched C₁-C₆ alkyl;

(iii) C₁-C₆ alkoxy;

(iv) amino;

(v) alkylamino;

(vi) C₁-C₄ dialkylamino;

(vii) nitro;

(viii) hydroxyl;

(ix) halogen, where “halogen” encompasses chloro, fluoro, bromo, and iodo;

R⁵, R⁶, R⁷, R⁸, and R⁹ are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo; and

n is an integer from 1 to 5.

In some embodiments, the peptide is defined by the formula:

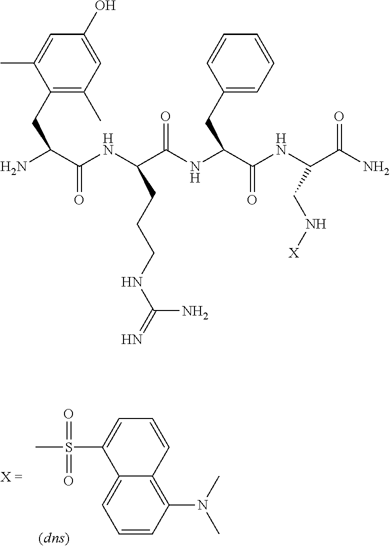

also represented as 2′,6′-Dmt-D-Arg-Phe-(dns)Dap-NH₂, where (dns)Dap is β-dansyl-L-α,β-diaminopropionic acid (SS-17).

In some embodiments, the peptide is defined by the formula:

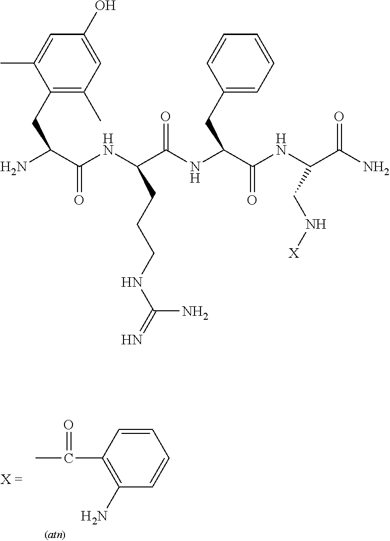

also represented as 2′,6′-Dmt-D-Arg-Phe-(atn)Dap-NH₂ where (atn)Dap is β-anthraniloyl-L-α,β-diaminopropionic acid (SS-19). SS-19 is also referred to as [atn]SS-02.

In a particular embodiment, R¹ and R² are hydrogen; R³ and R⁴ are methyl; R⁵, R⁶, R⁷, R⁸, and R⁹ are all hydrogen; and n is 4.

In one embodiment, the aromatic-cationic peptides have a core structural motif of alternating aromatic and cationic amino acids. For example, the peptide may be a tetrapeptide defined by any of formulas III to VI set forth below:

Aromatic-Cationic-Aromatic-Cationic (Formula III)

Cationic-Aromatic-Cationic-Aromatic (Formula IV)

Aromatic-Aromatic-Cationic-Cationic (Formula V)

Cationic-Cationic-Aromatic-Aromatic (Formula VI)

wherein, Aromatic is a residue selected from the group consisting of: Phe (F), Tyr (Y), Trp (W), and Cyclohexylalanine (Cha); and Cationic is a residue selected from the group consisting of: Arg (R), Lys (K), Norleucine (Nle), and 2-amino-heptanoic acid (Ahe).

In some embodiments, the aromatic-cationic peptides described herein comprise all levorotatory (L) amino acids.

In one embodiment, the aromatic-cationic peptide has

1. at least one net positive charge;

2. a minimum of three amino acids;

3. a maximum of about twenty amino acids;

4. a relationship between the minimum number of net positive charges (pₘ) and the total number of amino acid residues (r) wherein 3pₘ is the largest number that is less than or equal to r+1; and

5. a relationship between the minimum number of aromatic groups (a) and the total number of net positive charges (pₜ) wherein 2a is the largest number that is less than or equal to pₜ+1, except that when a is 1, pₜ may also be 1.

In another embodiment, the present technology provides a method for reducing the number of mitochondria undergoing a mitochondrial permeability transition (MPT), or preventing mitochondrial permeability transitioning in a removed organ of a mammal or treating or ameliorating symptoms, conditions or diseases characterized by Aβ-induced mitochondrial dysfunction. The method comprises administering to the removed organ an effective amount of an aromatic-cationic peptide having:

at least one net positive charge;

a minimum of three amino acids;

a maximum of about twenty amino acids;

a relationship between the minimum number of net positive charges (pₘ) and the total number of amino acid residues (r) wherein 3pₘ is the largest number that is less than or equal to r+1; and

a relationship between the minimum number of aromatic groups (a) and the total number of net positive charges (pₜ) wherein 2a is the largest number that is less than or equal to pₜ+1, except that when a is 1, pₜ may also be 1.

In yet another embodiment, the present technology provides a method of reducing the number of mitochondria undergoing mitochondrial permeability transition (MPT), or preventing mitochondria permeability transitioning in a mammal in need thereof, or treating or ameliorating symptoms, conditions or diseases characterized by Aβ-induced mitochondrial dysfunction. The method comprises administering to the mammal an effective amount of an aromatic-cationic peptide having:

at least one net positive charge;

a minimum of three amino acids;

a maximum of about twenty amino acids;

a relationship between the minimum number of net positive charges (pₘ) and the total number of amino acid residues (r) wherein 3 pₘ is the largest number that is less than or equal to r+1; and

a relationship between the minimum number of aromatic groups (a) and the total number of net positive charges (pₜ) wherein 3a is the largest number that is less than or equal to pₜ+1, except that when a is 1, pₜ may also be 1.

Aromatic-cationic peptides include, but are not limited to, the following illustrative peptides:
H-Phe-D-Arg Phe-Lys-Cys-NH₂
D-Arg-Dmt-Lys-Trp-NH₂;
D-Arg-Trp-Lys-Trp-NH₂;
D-Arg-Dmt-Lys-Phe-Met-NH₂;
H-D-Arg-Dmt-Lys(N^{α}Me)-Phe-NH₂;
H-D-Arg-Dmt-Lys-Phe(NMe)NH₂;
H-D-Arg-Dmt-Lys(N^{α}Me)-Phe(NMe)NH₂;
H-D-Arg(N^{α}Me)-Dmt(NMe)-Lys(N^{α}Me)-Phe(NMe)NH₂;
D-Arg-Dmt-Lys-Phe-Lys-Trp-NH₂;
D-Arg-Dmt-Lys-Dmt-Lys-Trp-NH₂;
D-Arg-Dmt-Lys-Phe-Lys-Met-NH₂;
D-Arg-Dmt-Lys-Dmt-Lys-Met-NH₂;
H-D-Arg-Dmt-Lys-Phe-Sar-Gly-Cys-NH₂;
H-D-Arg-Ψ[CH₂—NH]Dmt-Lys-Phe-NH₂;
H-D-Arg-Dmt-Ψ[CH₂—NH]Lys-Phe-NH₂;
H-D-Arg-Dmt-LysΨ[CH₂—NH]Phe-NH₂; and
H-D-Arg-Dmt-Ψ[CH₂—NH]Lys-Ψ[CH₂—NH]Phe-NH₂,
Tyr-D-Arg-Phe-Lys-NH₂,
2′,6′-Dmt-D-Arg-Phe-Lys-NH₂,
Phe-D-Arg-Phe-Lys-NH₂,
Phe-D-Arg-Dmt-Lys-NH₂,
D-Arg-2′6′mt-Lys-Phe-NH₂,
H-Phe-D-Arg-Phe-Lys-Cys-NH₂,
Lys-D-Arg-Tyr-NH₂,
D-Tyr-Trp-Lys-NH₂,
Trp-D-Lys-Tyr-Arg-NH₂,
Tyr-His-D-Gly-Met,
Tyr-D-Arg-Phe-Lys-Glu-NH₂,
Met-Tyr-D-Lys-Phe-Arg,
D-His-Glu-Lys-Tyr-D-Phe-Arg,
Lys-D-Gln-Tyr-Arg-D-Phe-Trp-NH₂,
Phe-D-Arg-Lys-Trp-Tyr-D-Arg-His,
Gly-D-Phe-Lys-Tyr-His-D-Arg-Tyr-NH₂,
Val-D-Lys-His-Tyr-D-Phe-Ser-Tyr-Arg-NH₂,
Trp-Lys-Phe-D-Asp-Arg-Tyr-D-His-Lys,
Lys-Trp-D-Tyr-Arg-Asn-Phe-Tyr-D-His-NH₂,
Thr-Gly-Tyr-Arg-D-His-Phe-Trp-D-His-Lys,
Asp-D-Trp-Lys-Tyr-D-His-Phe-Arg-D-Gly-Lys-NH₂,
D-His-Lys-Tyr-D-Phe-Glu-D-Asp-D-His-D-Lys-Arg-Trp-NH₂,
Ala-D-Phe-D-Arg-Tyr-Lys-D-Trp-His-D-Tyr-Gly-Phe,
Tyr-D-His-Phe-D-Arg-Asp-Lys-D-Arg-His-Trp-D-His-Phe,
Phe-Phe-D-Tyr-Arg-Glu-Asp-D-Lys-Arg-D-Arg-His-Phe-NH₂,
Phe-Tyr-Lys-D-Arg-Trp-His-D-Lys-D-Lys-Glu-Arg-D-Tyr-Thr,
Tyr-Asp-D-Lys-Tyr-Phe-D-Lys-D-Arg-Phe-Pro-D-Tyr-His-Lys,
Glu-Arg-D-Lys-Tyr-D-Val-Phe-D-His-Trp-Arg-D-Gly-Tyr-Arg-D-Met-NH₂,
Arg-D-Leu-D-Tyr-Phe-Lys-Glu-D-Lys-Arg-D-Trp-Lys-D-Phe-Tyr-D-Arg-Gly,
D-Glu-Asp-Lys-D-Arg-D-His-Phe-Phe-D-Val-Tyr-Arg-Tyr-D-Tyr-Arg-His-Phe-NH₂,
Asp-Arg-D-Phe-Cys-Phe-D-Arg-D-Lys-Tyr-Arg-D-Tyr-Trp-D-His-Tyr-D-Phe-Lys-Phe,
His-Tyr-D-Arg-Trp-Lys-Phe-D-Asp-Ala-Arg-Cys-D-Tyr-His-Phe-D-Lys-Tyr-His-Ser-NH₂,
Gly-Ala-Lys-Phe-D-Lys-Glu-Arg-Tyr-His-D-Arg-D-Arg-Asp-Tyr-Trp-D-His-Trp-His-D-Lys-Asp, and
Thr-Tyr-Arg-D-Lys-Trp-Tyr-Glu-Asp-D-Lys-D-Arg-His-Phe-D-Tyr-Gly-Val-Ile-D-His-Arg-Tyr-Lys-NH₂;
Dmt-D-Arg-Phe-(atn)Dap-NH₂, where (atn)Dap is β-anthraniloyl-L-α,β-diaminopropionic acid;
Dmt-D-Arg-Phe-(dns)Dap-NH₂ where (dns)Dap is β-dansyl-L-α,β-diaminopropionic acid;
Dmt-D-Arg-Ald-Lys-NH₂, where Ald is β-(6′-dimethylamino-2′-naphthoyl)alanine;
Dmt-D-Arg-Phe-Lys-Aid-NH₂, where Ald is β-(6′-dimethylamino-2′-naphthoyl)alanine and D-Arg-Tyr-Lys-Phe-NH₂; and
D-Arg-Tyr-Lys-Phe-NH₂.

In some embodiments, peptides useful in the methods of the present technology are those peptides which have a tyrosine residue or a tyrosine derivative. In some embodiments, derivatives of tyrosine include 2′-methyltyrosine (Mmt); 2′,6′-dimethyltyrosine (2′6′Dmt); 3′,5′-dimethyltyrosine (3′5′Dmt); N,2′,6′-trimethyltyrosine (Tmt); and 2′-hydroxy-6′-methyltryosine (Hmt).

In one embodiment, the peptide has the formula Tyr-D-Arg-Phe-Lys-NH₂ (referred to herein as SS-01). SS-01 has a net positive charge of three, contributed by the amino acids tyrosine, arginine, and lysine and has two aromatic groups contributed by the amino acids phenylalanine and tyrosine. The tyrosine of SS-01 can be a modified derivative of tyrosine such as in 2′,6′-dimethyltyrosine to produce the compound having the formula 2′,6′-Dmt-D-Arg-Phe-Lys-NH₂ (referred to herein as SS-02).

In a suitable embodiment, the amino acid residue at the N-terminus is arginine. An example of such a peptide is D-Arg-2′,6′ Dmt-Lys-Phe-NH₂ (referred to herein as SS-31).

In another embodiment, the amino acid at the N-terminus is phenylalanine or its derivative. In some embodiments, derivatives of phenylalanine include 2′-methylphenylalanine (Mmp), 2′,6′-dimethylphenylalanine (Dmp), N,2′,6′-trimethylphenylalanine (Tmp), and 2′-hydroxy-6′-methylphenylalanine (Hmp). An example of such a peptide is Phe-D-Arg-Phe-Lys-NH₂ (referred to herein as SS-20). In one embodiment, the amino acid sequence of SS-02 is rearranged such that Dmt is not at the N-terminus. An example of such an aromatic-cationic peptide has the formula D-Arg-2′,6′-Dmt-Lys-Phe-NH₂ (SS-31).

In yet another embodiment, the aromatic-cationic peptide has the formula Phe-D-Arg-Dmt-Lys-NH₂(referred to herein as SS-30). Alternatively, the N-terminal phenylalanine can be a derivative of phenylalanine such as 2′,6′-dimethylphenylalanine (2′6′Dmp). SS-01 containing 2′,6′-dimethylphenylalanine at amino acid position one has the formula 2′,6′-Dmp-D-Arg-Dmt-Lys-NH₂.

In some embodiments, the aromatic cationic peptide comprises 2′,6′-Dmt-D-Arg-Phe-(atn)Dap-NH₂ (SS-19), where (atn)Dap is β-anthraniloyl-L-α,β-diaminopropionic acid, 2′,6′-Dmt-D-Arg-Ald-Lys-NH₂ (SS-36), where Ald is β-(6′-dimethylamino-2′-naphthoyl)alanine, 2′,6′-Dmt-D-Arg-Phe-Lys-Ald-NH₂ (SS-37), where Ald is β-(6′-dimethylamino-2′-naphthoyl)alanine, D-Arg-Tyr-Lys-Phe-NH₂ (SPI-231), and 2′,6′-Dmt-D-Arg-Phe-(dns)Dap-NH₂ where (dns)Dap is β-dansyl-L-α,β-diaminopropionic acid (SS-17).

The peptides mentioned herein and their derivatives can further include functional analogs. A peptide is considered a functional analog if the analog has the same function as the stated peptide. The analog may, for example, be a substitution variant of a peptide, wherein one or more amino acids are substituted by another amino acid. Suitable substitution variants of the peptides include conservative amino acid substitutions. Amino acids may be grouped according to their physicochemical characteristics as follows:

(a) Non-polar amino acids: Ala(A) Ser(S) Thr(T) Pro(P) Gly(G) Cys (C);

(b) Acidic amino acids: Asn(N) Asp(D) Glu(E) Gln(Q);

(c) Basic amino acids: His(H) Arg(R) Lys(K);

(d) Hydrophobic amino acids: Met(M) Leu(L) Ile(I) Val(V); and

(e) Aromatic amino acids: Phe(F) Tyr(Y) Trp(W) His (H).

Substitutions of an amino acid in a peptide by another amino acid in the same group is referred to as a conservative substitution and may preserve the physicochemical characteristics of the original peptide. In contrast, substitutions of an amino acid in a peptide by another amino acid in a different group are generally more likely to alter the characteristics of the original peptide. Non-limiting examples of analogs useful in the practice of the present technology include, but are not limited to, the aromatic-cationic peptides shown in Table 5.

TABLE 5
Examples of Peptide Analogs

| Amino | Amino | Amino | Amino | Amino | Amino | Amino | |
|---|---|---|---|---|---|---|---|
| Acid | Acid | Acid | Acid | Acid | Acid | Acid | C-Terminal |
| Position 1 | Position 2 | Position 3 | Position 4 | Position 5 | Position 6 | Position 7 | Modification |
| D-Arg | Dmt | Lys | Phe | | | | NH₂ |
| D-Arg | Dmt | Phe | Lys | | | | NH₂ |
| D-Arg | Phe | Lys | Dmt | | | | NH₂ |
| D-Arg | Phe | Dmt | Lys | | | | NH₂ |
| D-Arg | Lys | Dmt | Phe | | | | NH₂ |
| D-Arg | Lys | Phe | Dmt | | | | NH₂ |
| D-Arg | Dmt | Lys | Phe | Cys | | | NH₂ |
| D-Arg | Dmt | Lys | Phe | Glu | Cys | Gly | NH₂ |
| D-Arg | Dmt | Lys | Phe | Ser | Cys | | NH₂ |
| D-Arg | Dmt | Lys | Phe | Gly | Cys | | NH₂ |
| Phe | Lys | Dmt | D-Arg | | | | NH₂ |
| Phe | Lys | D-Arg | Dmt | | | | NH₂ |
| Phe | D-Arg | Phe | Lys | | | | NH₂ |
| Phe | D-Arg | Phe | Lys | Cys | | | NH₂ |
| Phe | D-Arg | Phe | Lys | Glu | Cys | Gly | NH₂ |
| Phe | D-Arg | Phe | Lys | Ser | Cys | | NH₂ |
| Phe | D-Arg | Phe | Lys | Gly | Cys | | NH₂ |
| Phe | D-Arg | Dmt | Lys | | | | NH₂ |
| Phe | D-Arg | Dmt | Lys | Cys | | | NH₂ |
| Phe | D-Arg | Dmt | Lys | Glu | Cys | Gly | NH₂ |
| Phe | D-Arg | Dmt | Lys | Ser | Cys | | NH₂ |
| Phe | D-Arg | Dmt | Lys | Gly | Cys | | NH₂ |
| Phe | D-Arg | Lys | Dmt | | | | NH₂ |
| Phe | Dmt | D-Arg | Lys | | | | NH₂ |
| Phe | Dmt | Lys | D-Arg | | | | NH₂ |
| Lys | Phe | D-Arg | Dmt | | | | NH₂ |
| Lys | Phe | Dmt | D-Arg | | | | NH₂ |
| Lys | Dmt | D-Arg | Phe | | | | NH₂ |
| Lys | Dmt | Phe | D-Arg | | | | NH₂ |
| Lys | D-Arg | Phe | Dmt | | | | NH₂ |
| Lys | D-Arg | Dmt | Phe | | | | NH₂ |
| D-Arg | Dmt | D-Arg | Phe | | | | NH₂ |
| D-Arg | Dmt | D-Arg | Dmt | | | | NH₂ |
| D-Arg | Dmt | D-Arg | Tyr | | | | NH₂ |
| D-Arg | Dmt | D-Arg | Trp | | | | NH₂ |
| Trp | D-Arg | Phe | Lys | | | | NH₂ |
| Trp | D-Arg | Tyr | Lys | | | | NH₂ |
| Trp | D-Arg | Trp | Lys | | | | NH₂ |
| Trp | D-Arg | Dmt | Lys | | | | NH₂ |
| D-Arg | Trp | Lys | Phe | | | | NH₂ |
| D-Arg | Trp | Phe | Lys | | | | NH₂ |
| D-Arg | Trp | Lys | Dmt | | | | NH₂ |
| D-Arg | Trp | Dmt | Lys | | | | NH₂ |
| D-Arg | Lys | Trp | Phe | | | | NH₂ |
| D-Arg | Lys | Trp | Dmt | | | | NH₂ |
| Cha | D-Arg | Phe | Lys | | | | NH₂ |
| Ala | D-Arg | Phe | Lys | | | | NH₂ |

Cha = cyclohexylalanine

Under certain circumstances, it may be advantageous to use a peptide that also has opioid receptor agonist activity. Examples of analogs useful in the practice of the present technology include, but are not limited to, the aromatic-cationic peptides shown in Table 6.

TABLE 6
Peptide Analogs with Opioid Receptor Agonist Activity

| | | | | Amino | |
|---|---|---|---|---|---|
| Amino | Amino | Amino | | Acid | |
| Acid | Acid | Acid | Amino Acid | Position 5 | C-Terminal |
| Position 1 | Position 2 | Position 3 | Position 4 | (if present) | Modification |
| Tyr | D-Arg | Phe | Lys | | NH₂ |
| Tyr | D-Arg | Phe | Orn | | NH₂ |
| Tyr | D-Arg | Phe | Dab | | NH₂ |
| Tyr | D-Arg | Phe | Dap | | NH₂ |
| Tyr | D-Arg | Phe | Lys | Cys | NH₂ |
| 2′6′Dmt | D-Arg | Phe | Lys | | NH₂ |
| 2′6′Dmt | D-Arg | Phe | Lys | Cys | NH₂ |
| 2′6′Dmt | D-Arg | Phe | Lys- | | NH₂ |
| | | | NH(CH₂)₂—NH- | | |
| | | | dns | | |
| 2′6′Dmt | D-Arg | Phe | Lys- | | NH₂ |
| | | | NH(CH₂)₂—NH- | | |
| | | | atn | | |
| 2′6′Dmt | D-Arg | Phe | dnsLys | | NH₂ |
| 2′6′Dmt | D-Cit | Phe | Lys | | NH₂ |
| 2′6′Dmt | D-Cit | Phe | Lys | Cys | NH₂ |
| 2′6′Dmt | D-Cit | Phe | Ahp | | NH₂ |
| 2′6′Dmt | D-Arg | Phe | Orn | | NH₂ |
| 2′6′Dmt | D-Arg | Phe | Dab | | NH₂ |
| 2′6′Dmt | D-Arg | Phe | Dap | | NH₂ |
| 2′6′Dmt | D-Arg | Phe | Ahp(2- | | NH₂ |
| | | | aminoheptanoic | | |
| | | | acid) | | |
| Bio- | D-Arg | Phe | Lys | | NH₂ |
| 2′6′Dmt | | | | | |
| 3′5′Dmt | D-Arg | Phe | Lys | | NH₂ |
| 3′5′Dmt | D-Arg | Phe | Orn | | NH₂ |
| 3′5′Dmt | D-Arg | Phe | Dab | | NH₂ |
| 3′5′Dmt | D-Arg | Phe | Dap | | NH₂ |
| Tyr | D-Arg | Tyr | Lys | | NH₂ |
| Tyr | D-Arg | Tyr | Orn | | NH₂ |
| Tyr | D-Arg | Tyr | Dab | | NH₂ |
| Tyr | D-Arg | Tyr | Dap | | NH₂ |
| 2′6′Dmt | D-Arg | Tyr | Lys | | NH₂ |
| 2′6′Dmt | D-Arg | Tyr | Orn | | NH₂ |
| 2′6′Dmt | D-Arg | Tyr | Dab | | NH₂ |
| 2′6′Dmt | D-Arg | Tyr | Dap | | NH₂ |
| 2′6′Dmt | D-Arg | 2′6′Dmt | Lys | | NH₂ |
| 2′6′Dmt | D-Arg | 2′6′Dmt | Orn | | NH₂ |
| 2′6′Dmt | D-Arg | 2′6′Dmt | Dab | | NH₂ |
| 2′6′Dmt | D-Arg | 2′6′Dmt | Dap | | NH₂ |
| 3′5′Dmt | D-Arg | 3′5′Dmt | Arg | | NH₂ |
| 3′5′Dmt | D-Arg | 3′5′Dmt | Lys | | NH₂ |
| 3′5′Dmt | D-Arg | 3′5′Dmt | Orn | | NH₂ |
| 3′5′Dmt | D-Arg | 3′5′Dmt | Dab | | NH₂ |
| 2′6′Dmt | D-Arg | 2′6′Dmt | Lys | Cys | NH₂ |
| Tyr | D-Lys | Phe | Dap | | NH₂ |
| Tyr | D-Lys | Phe | Arg | | NH₂ |
| Tyr | D-Lys | Phe | Arg | Cys | NH₂ |
| Tyr | D-Lys | Phe | Lys | | NH₂ |
| Tyr | D-Lys | Phe | Orn | | NH₂ |
| 2′6′Dmt | D-Lys | Phe | Dab | | NH₂ |
| 2′6′Dmt | D-Lys | Phe | Dap | | NH₂ |
| 2′6′Dmt | D-Lys | Phe | Arg | | NH₂ |
| 2′6′Dmt | D-Lys | Phe | Lys | | NH₂ |
| 3′5′Dmt | D-Lys | Phe | Orn | | NH₂ |
| 3′5′Dmt | D-Lys | Phe | Dab | | NH₂ |
| 3′5′Dmt | D-Lys | Phe | Dap | | NH₂ |
| 3′5′Dmt | D-Lys | Phe | Arg | | NH₂ |
| 3′5′Dmt | D-Lys | Phe | Arg | Cys | NH₂ |
| Tyr | D-Lys | Tyr | Lys | | NH₂ |
| Tyr | D-Lys | Tyr | Orn | | NH₂ |
| Tyr | D-Lys | Tyr | Dab | | NH₂ |
| Tyr | D-Lys | Tyr | Dap | | NH₂ |
| 2′6′Dmt | D-Lys | Tyr | Lys | | NH₂ |
| 2′6′Dmt | D-Lys | Tyr | Orn | | NH₂ |
| 2′6′Dmt | D-Lys | Tyr | Dab | | NH₂ |
| 2′6′Dmt | D-Lys | Tyr | Dap | | NH₂ |
| 2′6′Dmt | D-Lys | 2′6′Dmt | Lys | | NH₂ |
| 2′6′Dmt | D-Lys | 2′6′Dmt | Orn | | NH₂ |
| 2′6′Dmt | D-Lys | 2′6′Dmt | Dab | | NH₂ |
| 2′6′Dmt | D-Lys | 2′6′Dmt | Dap | | NH₂ |
| 2′6′Dmt | D-Arg | Phe | dnsDap | | NH₂ |
| 2′6′Dmt | D-Arg | Phe | atnDap | | NH₂ |
| 3′5′Dmt | D-Lys | 3′5′Dmt | Lys | | NH₂ |
| 3′5′Dmt | D-Lys | 3′5′Dmt | Orn | | NH₂ |
| 3′5′Dmt | D-Lys | 3′5′Dmt | Dab | | NH₂ |
| 3′5′Dmt | D-Lys | 3′5′Dmt | Dap | | NH₂ |
| Tyr | D-Lys | Phe | Arg | | NH₂ |
| Tyr | D-Orn | Phe | Arg | | NH₂ |
| Tyr | D-Dab | Phe | Arg | | NH₂ |
| Tyr | D-Dap | Phe | Arg | | NH₂ |
| 2′6′Dmt | D-Arg | Phe | Arg | | NH₂ |
| 2′6′Dmt | D-Lys | Phe | Arg | | NH₂ |
| 2′6′Dmt | D-Orn | Phe | Arg | | NH₂ |
| 2′6′Dmt | D-Dab | Phe | Arg | | NH₂ |
| 3′5′Dmt | D-Dap | Phe | Arg | | NH₂ |
| 3′5′Dmt | D-Arg | Phe | Arg | | NH₂ |
| 3′5′Dmt | D-Lys | Phe | Arg | | NH₂ |
| 3′5′Dmt | D-Orn | Phe | Arg | | NH₂ |
| Tyr | D-Lys | Tyr | Arg | | NH₂ |
| Tyr | D-Orn | Tyr | Arg | | NH₂ |
| Tyr | D-Dab | Tyr | Arg | | NH₂ |
| Tyr | D-Dap | Tyr | Arg | | NH₂ |
| 2′6′Dmt | D-Arg | 2′6′Dmt | Arg | | NH₂ |
| 2′6′Dmt | D-Lys | 2′6′Dmt | Arg | | NH₂ |
| 2′6′Dmt | D-Orn | 2′6′Dmt | Arg | | NH₂ |
| 2′6′Dmt | D-Dab | 2′6′Dmt | Arg | | NH₂ |
| 3′5′Dmt | D-Dap | 3′5′Dmt | Arg | | NH₂ |
| 3′5′Dmt | D-Arg | 3′5′Dmt | Arg | | NH₂ |
| 3′5′Dmt | D-Lys | 3′5′Dmt | Arg | | NH₂ |
| 3′5′Dmt | D-Orn | 3′5′Dmt | Arg | | NH₂ |
| Mmt | D-Arg | Phe | Lys | | NH₂ |
| Mmt | D-Arg | Phe | Orn | | NH₂ |
| Mmt | D-Arg | Phe | Dab | | NH₂ |
| Mmt | D-Arg | Phe | Dap | | NH₂ |
| Tmt | D-Arg | Phe | Lys | | NH₂ |
| Tmt | D-Arg | Phe | Orn | | NH₂ |
| Tmt | D-Arg | Phe | Dab | | NH₂ |
| Tmt | D-Arg | Phe | Dap | | NH₂ |
| Hmt | D-Arg | Phe | Lys | | NH₂ |
| Hmt | D-Arg | Phe | Orn | | NH₂ |
| Hmt | D-Arg | Phe | Dab | | NH₂ |
| Hmt | D-Arg | Phe | Dap | | NH₂ |
| Mmt | D-Lys | Phe | Lys | | NH₂ |
| Mmt | D-Lys | Phe | Orn | | NH₂ |
| Mmt | D-Lys | Phe | Dab | | NH₂ |
| Mmt | D-Lys | Phe | Dap | | NH₂ |
| Mmt | D-Lys | Phe | Arg | | NH₂ |
| Tmt | D-Lys | Phe | Lys | | NH₂ |
| Tmt | D-Lys | Phe | Orn | | NH₂ |
| Tmt | D-Lys | Phe | Dab | | NH₂ |
| Tmt | D-Lys | Phe | Dap | | NH₂ |
| Tmt | D-Lys | Phe | Arg | | NH₂ |
| Hmt | D-Lys | Phe | Lys | | NH₂ |
| Hmt | D-Lys | Phe | Orn | | NH₂ |
| Hmt | D-Lys | Phe | Dab | | NH₂ |
| Hmt | D-Lys | Phe | Dap | | NH₂ |
| Hmt | D-Lys | Phe | Arg | | NH₂ |
| Mmt | D-Lys | Phe | Arg | | NH₂ |
| Mmt | D-Orn | Phe | Arg | | NH₂ |
| Mmt | D-Dab | Phe | Arg | | NH₂ |
| Mmt | D-Dap | Phe | Arg | | NH₂ |
| Mmt | D-Arg | Phe | Arg | | NH₂ |
| Tmt | D-Lys | Phe | Arg | | NH₂ |
| Tmt | D-Orn | Phe | Arg | | NH₂ |
| Tmt | D-Dab | Phe | Arg | | NH₂ |
| Tmt | D-Dap | Phe | Arg | | NH₂ |
| Tmt | D-Arg | Phe | Arg | | NH₂ |
| Hmt | D-Lys | Phe | Arg | | NH₂ |
| Hmt | D-Orn | Phe | Arg | | NH₂ |
| Hmt | D-Dab | Phe | Arg | | NH₂ |
| Hmt | D-Dap | Phe | Arg | | NH₂ |
| Hmt | D-Arg | Phe | Arg | | NH₂ |

Dab = diaminobutyric,
Dap = diaminopropionic acid,
Dmt = dimethyltyrosine,
Mmt = 2′-methyltyrosine,
Tmt = N, 2′,6′-trimethyltyrosine,
Hmt = 2′-hydroxy,6′-methyltyrosine,
dnsDap = β-dansyl-L-α,β-diaminopropionic acid,
atnDap = β-anthraniloyl-L-α,β-diaminopropionic acid,
Bio = biotin

Additional peptides having opioid receptor agonist activity include 2′,6′-Dmt-D-Arg-Ald-Lys-NH₂ (SS-36), where Ald is β-(6′-dimethylamino-2′-naphthoyl)alanine, and 2′,6′-Dmt-D-Arg-Phe-Lys-Ald-NH₂ (SS-37), where Ald is β-(6′-dimethylamino-2′-naphthoyl)alanine.

Peptides which have mu-opioid receptor agonist activity are typically those peptides which have a tyrosine residue or a tyrosine derivative at the N-terminus (i.e., the first amino acid position). Suitable derivatives of tyrosine include 2′-methyltyrosine (Mmt); 2′,6′-dimethyltyrosine (2′6′-Dmt); 3′,5′-dimethyltyrosine (3′5′Dmt); N,2′,6′-trimethyltyrosine (Tmt); and 2′-hydroxy-6′-methyltryosine (Hmt).

Peptides that do not have mu-opioid receptor agonist activity generally do not have a tyrosine residue or a derivative of tyrosine at the N-terminus (i.e., amino acid position 1). The amino acid at the N-terminus can be any naturally occurring or non-naturally occurring amino acid other than tyrosine. In one embodiment, the amino acid at the N-terminus is phenylalanine or its derivative. Exemplary derivatives of phenylalanine include 2′-methylphenylalanine (Mmp), 2′,6′-dimethylphenylalanine (2′,6′-Dmp), N,2′,6′-trimethylphenylalanine (Tmp), and 2′-hydroxy-6′-methylphenylalanine (Hmp).

The amino acids of the peptides shown in Tables 5 and 6 may be in either the L- or the D-configuration.

In some embodiments, the aromatic-cationic peptides include at least one arginine and/or at least one lysine residue. In some embodiments, the arginine and/or lysine residue serves as an electron acceptor and participates in proton coupled electron transport. Additionally or alternatively, in some embodiments, the aromatic-cationic peptide comprises a sequence resulting in a “charge-ring-charge-ring” configuration such as exists in D-Arg-2′,6′-Dmt-Lys-Phe-NH₂. Additionally or alternatively, in some embodiments the aromatic-cationic peptides include thiol-containing residues, such as cysteine and methionine. In some embodiments, peptides including thiol-containing residues directly donate electrons and reduce cytochrome c. In some embodiments, the aromatic-cationic peptides include a cysteine at the N- and/or at the C-terminus of the peptide.

In some embodiments, the aromatic-cationic peptides described herein comprise all levorotatory (L) amino acids.

In some embodiments, the aromatic-cationic peptides described herein are synthesized with aladan, e.g., D-Arg-2′6′-Dmt-Lys-Ald-NH₂([ald]SS-31).

The peptides may be synthesized by any of the methods well known in the art. Suitable methods for chemically synthesizing the protein include, for example, those described by Stuart and Young in Solid Phase Peptide Synthesis, Second Edition, Pierce Chemical Company (1984), and in Methods Enzymol., 289, Academic Press, Inc., New York (1997).

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol. (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively.

### Use of the Aromatic-Cationic Peptides to Prevent, Ameliorate, or Treat Osteoarthritis

General. In some embodiments, the methods disclosed herein provide therapies for the prevention, amelioration or treatment of osteoarthritis (OA) or post-traumatic osteoarthritis (PTOA) and/or one or more symptoms of OA or PTOA comprising administering an effective amount of an aromatic-cationic peptide or a pharmaceutically acceptable salt thereof, such as acetate, tartrate salt, or trifluoroacetate salt.

The aromatic-cationic peptides described herein, such as D-Arg-2′,6′-Dmt-Lys-Phe-NH₂, or pharmaceutically acceptable salts thereof, such as acetate salt, tartrate salt, or trifluoroacetate salt, are useful to prevent or treat OA or PTOA. Specifically, the disclosure provides for both prophylactic and therapeutic methods of treating a subject having or suspected of having OA or PTOA. By way of example, but not by way of limitation, in some embodiments, the disclosure provides for both prophylactic and therapeutic methods of treating a subject exhibiting cartilage degeneration and/or chondrocyte death in joint(s) caused by a mechanical injury. Accordingly, in some embodiments, the present methods provide for the prevention and/or treatment of OA or PTOA in a subject by administering an effective amount of an aromatic-cationic peptide to a subject in need thereof to reduce cartilage degeneration and/or chondrocyte death in the effected joint(s) of the subject. In some embodiments, the present technology relates to the treatment, amelioration or prevention of OA or PTOA in mammals through administration of therapeutically effective amounts of aromatic-cationic peptides as disclosed herein, such as D-Arg-2′,6′-Dmt-Lys-Phe-NH₂, or pharmaceutically acceptable salts thereof, such as acetate salt, tartrate salt, or trifluoroacetate salt, to subjects in need thereof. In some embodiments, the OA or PTOA is present in a joint, shoulder, hand, foot, ankle, toe, hip, spine, jaw, or knee.

### Prophylactic and Therapeutic Uses of Peptide

In some embodiments, at least one aromatic-cationic peptide, such as D-Arg-2′,6′-Dmt-Lys-Phe-NH₂, or a pharmaceutically acceptable salt thereof, such as acetate, tartrate salt, or trifluoroacetate salt, described herein are useful for preventing or treating OA or PTOA. Specifically, the disclosure provides for both prophylactic and therapeutic methods of treating a subject suffering from, at risk of, or susceptible to OA or PTOA. Accordingly, the present methods provide for the prevention and/or treatment of OA or PTOA in a subject by administering an effective amount of at least one aromatic peptide, such as D-Arg-2′,6′-Dmt-Lys-Phe-NH₂, or a pharmaceutically acceptable salt thereof, such as acetate, tartrate salt, or trifluoroacetate salt, to a subject in need thereof. In some embodiments, a subject is administered at least one aromatic-cationic peptide in an effort to prevent, treat, or ameliorate OA or PTOA.

In some embodiments, administration of an effective amount of at least one aromatic-cationic peptide, such as D-Arg-2′,6′-Dmt-Lys-Phe-NH₂, or a pharmaceutically acceptable salt thereof, such as acetate, tartrate salt, or trifluoroacetate salt, alleviates or eliminates one or more symptoms of OA or PTOA in a subject for therapeutic purposes. In therapeutic applications, compositions or medicaments are administered to a subject suspected of, or already suffering from OA or PTOA in an amount sufficient to cure, or at least partially arrest, the symptoms of the OA or PTOA, including its complications and intermediate pathological phenotypes in development of the disease. In some embodiments, administration of an effective amount of at least one aromatic-cationic peptide, such as D-Arg-2′,6′-Dmt-Lys-Phe-NH₂, or a pharmaceutically acceptable salt thereof, such as acetate, tartrate salt, or trifluoroacetate salt, to a subject modulates one or more signs or symptoms of OA or PTOA. By way of example, but not by way of limitation, signs and symptoms of OA or PTOA include, but are not limited to, joint pain; joint swelling; joint clicking; joint cracking and/or creaking; joint stiffness; limited range of motion in a joint; pain in the groin, buttocks, inside knee, or thigh; grating or scraping sensation during movement of a knee; pain or tenderness in a toe joint; and swelling in ankles or toes. As such, the disclosure provides methods of treating an individual afflicted with OA or PTOA. Subjects suffering from OA or PTOA can be identified by, e.g., any diagnostic or prognostic assays known in the art.

In prophylactic applications, pharmaceutical compositions or medicaments of an effective amount of at least one aromatic-cationic peptide, such as D-Arg-2′,6′-Dmt-Lys-Phe-NH₂, or a pharmaceutically acceptable salt thereof, such as acetate, tartrate salt, or trifluoroacetate salt, are administered to a subject susceptible to, or otherwise at risk of a disease or condition in an amount sufficient to eliminate or reduce the risk, lessen the severity, or delay the onset of OA or PTOA, including biochemical, histologic and/or behavioral symptoms of the disease, its complications, and intermediate pathological phenotypes presenting during development of the disease. Subjects at risk for OA or PTOA can be identified by, e.g., any diagnostic or prognostic assays known in the art. In some embodiments, administration of at least one aromatic-cationic peptide, such as D-Arg-2′,6′-Dmt-Lys-Phe-NH₂, or a pharmaceutically acceptable salt thereof, such as acetate, tartrate salt, or trifluoroacetate salt, occurs prior to the manifestation of symptoms characteristic of the aberrancy, such that a disease or disorder is prevented or, alternatively, delayed in its progression. By way of example, but not by way of limitation, in some embodiments, administration of at least one aromatic-cationic peptide of the present technology, delays the onset or reduces one or more signs or symptoms of OA or PTOA, including, but not limited to, joint pain; joint swelling; joint clicking; joint cracking and/or creaking; joint stiffness; limited range of motion in a joint; pain in the groin, buttocks, inside knee, or thigh; grating or scraping sensation during movement of a knee; pain or tenderness in a toe joint; and swelling in ankles or toes. The appropriate compound can be determined based on screening assays described herein. By way of example, but not by way of limitation, subjects at risk for OA or PTOA include, but are not limited to, subjects that will have an intraarticular surgical procedure, subjects with a history of trauma, military personnel, athletes (e.g., in basketball, football, soccer, and rugby), and parachuters (e.g., base-jumpers and skydivers).

### Determination of the Biological Effect of the Aromatic-Cationic Peptides of the Present Technology

In various embodiments, suitable in vitro or in vivo assays are performed to determine the effect of a specific aromatic-cationic peptide-based therapeutic and whether its administration is indicated for treatment. In various embodiments, in vitro assays can be performed with representative animal models, to determine if a given aromatic-cationic peptide-based therapeutic exerts the desired effect in reducing cartilage degeneration and chondrocyte death. Compounds for use in therapy can be tested in suitable animal model systems including, but not limited to, rats, mice, chicken, cows, monkeys, rabbits, and the like, prior to testing in human subjects. Similarly, for in vivo testing, any of the animal model systems known in the art can be used prior to administration to human subjects.

Accordingly, in some embodiments, therapeutic and/or prophylactic treatment of subjects having OA or PTOA, with an aromatic-cationic peptide as disclosed herein, such as D-Arg-2′6′Dmt-Lys-Phe-NH₂ (SS-31) or a pharmaceutically acceptable salt thereof, such as acetate, tartrate salt, or trifluoroacetate salt, will reduce cartilage degeneration and/or chondrocyte death in the effected joint(s) of the subject, thereby ameliorating symptoms of OA or PTOA. Symptoms of OA or PTOA include, but are not limited to, joint pain, swelling of joint, clicking, cracking, and/or creaking of joints, stiff joints, limited range of motion in joint, pain in groin, buttocks, or inside knee or thigh, grating or scraping sensation during movement of knee, pain and tenderness in large joint at base of big toe, and swelling in ankles or toes.

### Modes of Administration and Effective Dosages

Any method known to those in the art for contacting a cell, organ or tissue with a peptide may be employed. Suitable methods include in vitro, ex vivo, or in vivo methods. In vivo methods typically include the administration of an aromatic-cationic peptide, such as those described above, to a mammal, suitably a human.

In some embodiments, the aromatic-cationic peptide is administered in vivo to animals (e.g., for veterinary treatments). In some embodiments, the animals are agricultural livestock, e.g., horses, cows, and pigs. In some embodiments, the animals are companion animals, e.g., cats and dogs.

When used in vivo for therapy, the aromatic-cationic peptides are administered to the subject in effective amounts (i.e., amounts that have desired therapeutic effect). The dose and dosage regimen will depend upon the degree of the condition in the subject, the characteristics of the particular aromatic-cationic peptide used, e.g., its therapeutic index, the subject, and the subject's history.

The effective amount may be determined during pre-clinical trials and clinical trials by methods familiar to physicians and clinicians. An effective amount of an aromatic-cationic peptide useful in the methods may be administered to a mammal in need thereof by any of a number of well-known methods for administering pharmaceutical compounds. The aromatic-cationic peptide may be administered systemically or locally.

The aromatic-cationic peptide may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a peptide contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like. In some embodiments, the salt is an acetate, tartrate salt, or trifluoroacetate salt.

The aromatic-cationic peptides of the present technology described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal, or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The aromatic-cationic peptide compositions can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, isotonic agents are included, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the aromatic-cationic peptides of the present technology can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of an aromatic-cationic peptide of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

An aromatic-cationic peptide of the present technology can be formulated in a carrier system. The carrier can be a colloidal system. The colloidal system can be a liposome, a phospholipid bilayer vehicle. In one embodiment, the therapeutic peptide is encapsulated in a liposome while maintaining peptide integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles, and viral vector systems.

The carrier can also be a polymer, e.g., a biodegradable, biocompatible polymer matrix. In one embodiment, the aromatic-cationic peptide of the present technology can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the aromatic-cationic peptides of the present technology are prepared with carriers that will protect the aromatic-cationic peptides of the present technology against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The aromatic-cationic peptides of the present technology can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi et al., Cancer Lett., 100:63-69 (1996), describes the use of fusogenic liposomes to deliver a protein to cells both in vivo and in vitro.

Dosage, toxicity and therapeutic efficacy of the aromatic-cationic peptide of the present technology can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, the aromatic-cationic peptides of the present technology exhibit high therapeutic indices. While aromatic-cationic peptides of the present technology that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED50 with little or no toxicity. The dosage may vary within this range depending upon the dosage form employed and the route of administration utilized. For any aromatic-cationic peptide of the present technology used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the aromatic-cationic peptides of the present technology, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of peptide ranges from 0.001-10,000 micrograms per kg body weight. In one embodiment, aromatic-cationic peptide concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, and until the subject shows partial or complete amelioration of symptoms of disease. Thereafter, the patient can be administered a prophylactic regime.

In some embodiments, a therapeutically effective amount of an aromatic-cationic peptide of the present technology may be defined as a concentration of peptide at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue. In some embodiments, the doses are administered by single daily or weekly administration, but may also include continuous administration (e.g., parenteral infusion or transdermal application). In some embodiments, the dosage of the aromatic-cationic peptide of the present technology is provided at a “low,” “mid,” or “high” dose level. In one embodiment, the low dose is provided from about 0.0001 to about 0.5 mg/kg/h, suitably from about 0.001 to about 0.1 mg/kg/h. In one embodiment, the mid-dose is provided from about 0.01 to about 1.0 mg/kg/h, suitably from about 0.01 to about 0.5 mg/kg/h. In one embodiment, the high dose is provided from about 0.5 to about 10 mg/kg/h, suitably from about 0.5 to about 2 mg/kg/h.

In some embodiments, a therapeutically effective amount of an aromatic-cationic peptide of the present technology is administered about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, or a time between any two of the preceding times after mechanical injury. In some embodiments, a therapeutically effective amount of an aromatic-cationic peptide of the present technology is administered between about 1 to 15 hours, 2 to 14 hours, 3 to 13 hours, 4 to 12 hours, 5 to 11 hours, 6 to 10 hours, or 7 to 9 hours after mechanical injury. In some embodiments, a therapeutically effective amount of an aromatic-cationic peptide of the present technology is administered between about 1 to 60 minutes, 5 to 55 minutes, 10 to 50 minutes, 15 to 45 minutes, 20 to 40 minutes, or 25 to 35 minutes after mechanical injury. In some embodiments, a therapeutically effective amount of an aromatic-cationic peptide of the present technology is administered immediately after mechanical injury.

In some embodiments, a subject in need thereof is administered a therapeutically effective amount of at least one aromatic-cationic peptide of the present technology after being diagnosed with OA or PTOA.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

### EXAMPLES

The present technology is further illustrated by the following examples, which should not be construed as limiting in any way. For each of the examples below, any aromatic-cationic peptide described herein could be used. By way of example, but not by limitation, the aromatic-cationic peptide used in the examples below could be 2′,6′-Dmt-D-Arg-Phe-Lys-NH₂, Phe-D-Arg-Phe-Lys-NH₂, or D-Arg-2′,6′-Dmt-Lys-Phe-NH₂.

### Example 1

### Acute Mitochondrial Dysfunction in Cartilage Following Mechanical Injury

### Methods

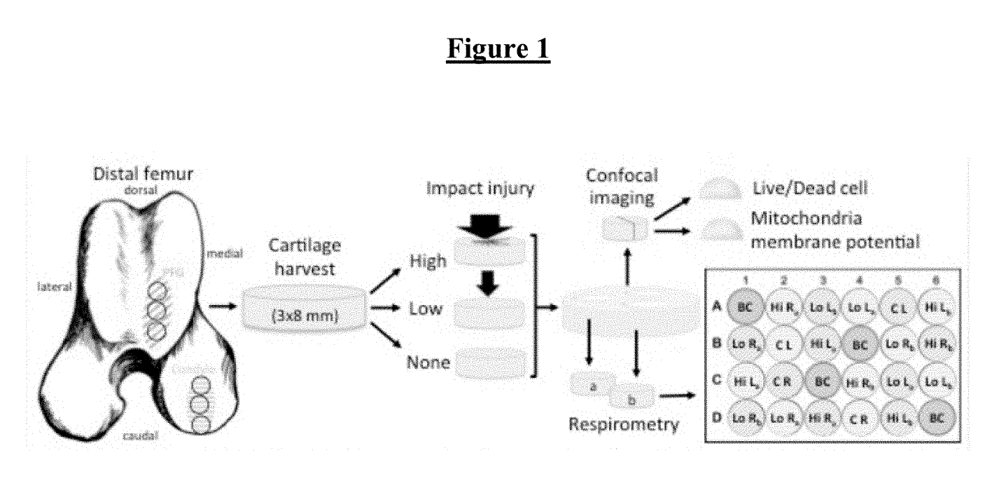

Cartilage was harvested from knees of neonatal bovids. Explants were subjected to unconfined compression (4-8 MPa peak stress; 5-10 GPa/s peak stress rate) using a validated sub-critical damage model. Explants were then divided for use in 3 assays (FIG. 1). Mitochondrial function was assessed in real time by microscale respirometry. Mitochondria membrane potential was measured by polarity-sensitive fluorescent staining. Chondrocyte viability was evaluated on confocal microscopy. Microscale respirometry was performed on explants loaded into a 24-well tissue capture microplate and analyzed in a Seahorse XF 24 analyzer. Glycolysis and oxidative phosphorylation were quantified every 8 minutes for a total of 245 minutes by measuring extracellular acidification (ECAR) and oxygen consumption rates (OCR), respectively. To measure specific indices of mitochondria function, a mitochondria stress test was performed by sequentially adding: 1) oligomycin, an ATP synthase inhibitor; 2) FCCP, a proton circuit uncoupler; and 3) rotenone+antimycin A (inhibitors of mitochondria complexes I and III) to determine ATP turnover, spare respiratory capacity, and proton leak across the inner mitochondria membrane, respectively. Relative mitochondria membrane potential was measured by the fluorescent intensity ratio of a polarity-insensitive mitochondria fluorescent probe (MitoTracker Green) to a polarity sensitive mitochondria marker (TMRM) on confocal microscopy.

### Results

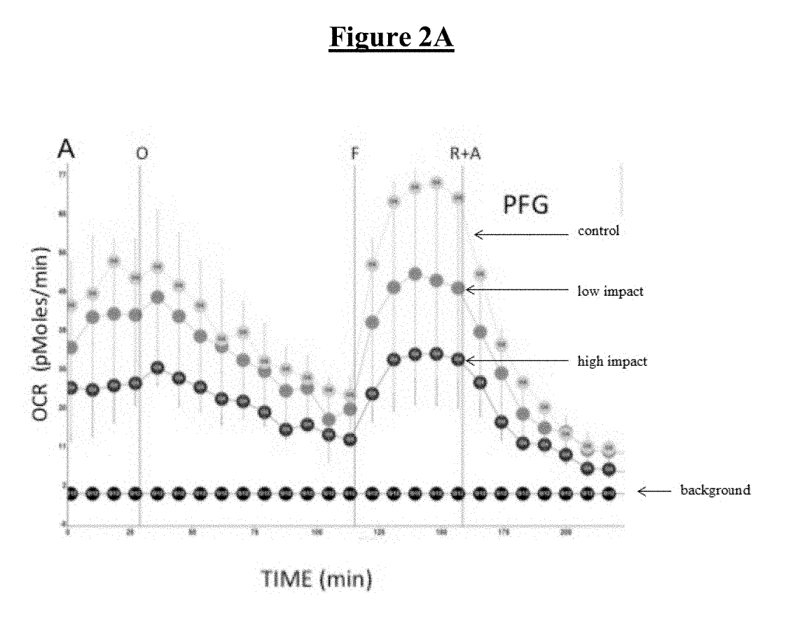

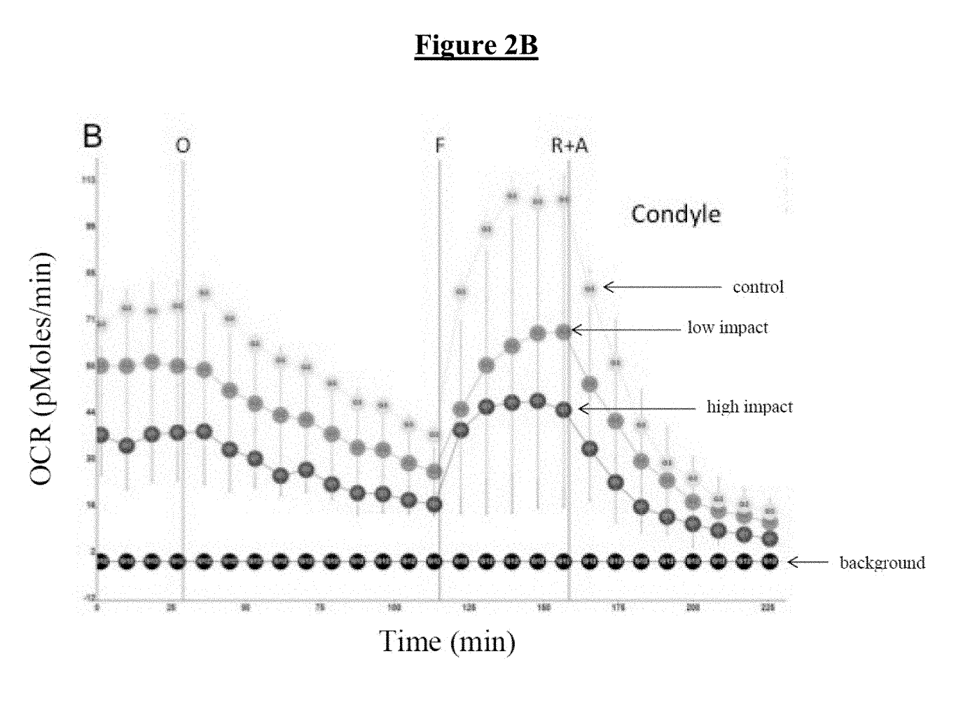

Baseline OCRs were higher in control samples than impacted samples and higher in cartilage from the femoral condyle than the patellofemoral groove (FIGS. 2A and 2B). Within two hours of injury, explants displayed impaired respiratory control in response to respiratory inhibitors (FIGS. 2A and 2B). Injured samples demonstrated an attenuated response to FCCP with a 61% (range 43-71) decrease in spare respiratory capacity.

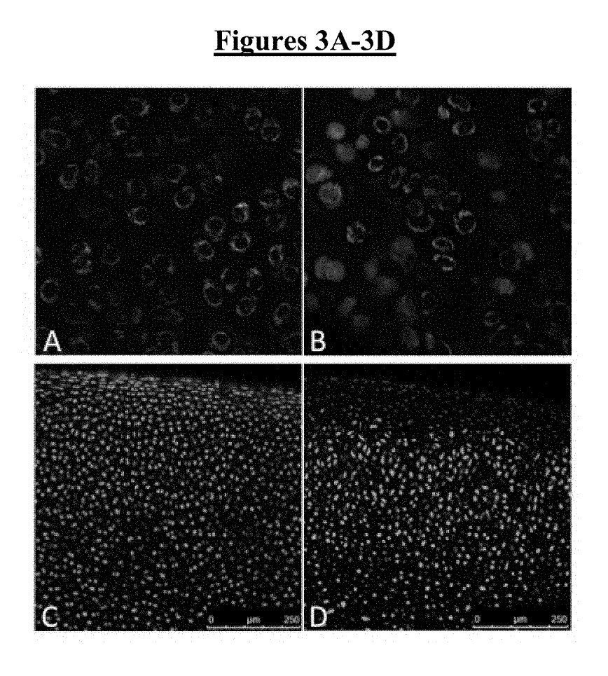

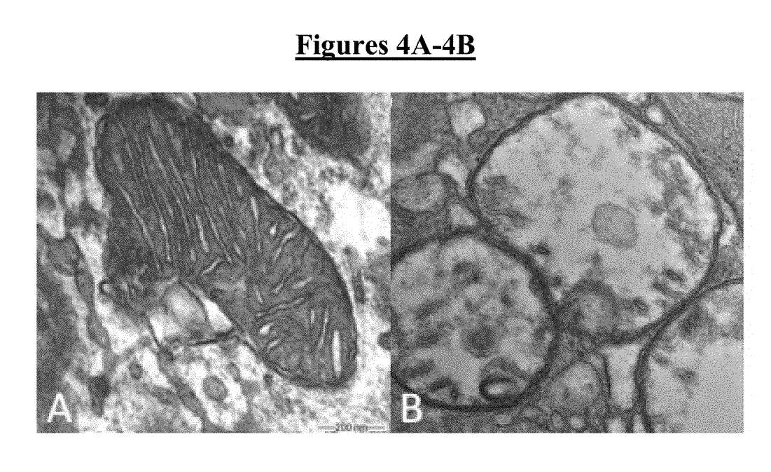

Significant differences in ECAR between groups were not detected. Cell viability was decreased in impacted samples by an average of 20% (range 5-38) versus non-impacted controls (FIG. 2A). Mitochondria membrane potential was decreased in impacted samples versus controls (FIG. 3), with a 34% (range 3-54) decrease in red:green (polarized mitochondria: all mitochondria) fluorescent intensity ratio after injury. Electron microscopy images of healthy mitochondria from uninjured cartilage and mitochondria from injured cartilage showed mitochondria swelling and loss of membrane folds in the mitochondria from injured cartilage (FIG. 4).

The data shows that mitochondria dysfunction is a peracute response of chondrocytes to mechanical injury. Over the described range of impact magnitudes, cartilage compression resulted in decreased basal respiration, compromised ATP turnover and reduced maximal respiratory capacity, which taken together indicate the inhibition of electron transport in the mitochondrial respiratory chain.

### Example 2

### D-Ar₂-2′,6′-Dmt-Lys-Phe-NH₂ (SS-31) Prevents Chondrocyte Death and Cartilage Degeneration Following Mechanical Injury

### Methods

Cartilage was harvested from the knee joints of 4 neonatal bovids (n=30 explants). Cartilage explants were subjected to unconfined compression (24.0±1.4 MPa peak stress; 53.8±5.3 GPa/s peak stress rate) using a validated single-impact subcritical damage model.

Cartilage explants were treated with 1 μM D-Arg-Dmt-Lys-Phe-NH₂ (SS-31) immediately after injury (T₀), one hour following injury (T₁), or 6 hours after injury (T₆), and then cultured for 7 days. Cartilage conditioned media (CCM) was sampled at T₀, T₁, T₆, and every 24 hours after injury (T₂₄₋₁₆₈) for 7 days.

Explants were stained with calcein AM and ethidium homodimer (for live and dead cells, respectively) to assess chondrocyte viability at T₂₄ and T₁₆₈ using confocal microscopy. Live, dead and total cell numbers were quantified in z-stacked digital images using a custom ImageJ macro. To quantify cell membrane damage, CCM was analyzed using a colorimetric lactate dehydrogenase (LDH) activity assay, and cumulative cell membrane damage over the 7-day incubation period was determined for each explant.

Cartilage matrix degradation was quantified by measuring GAG loss into the media via DMMB assay.

### Results

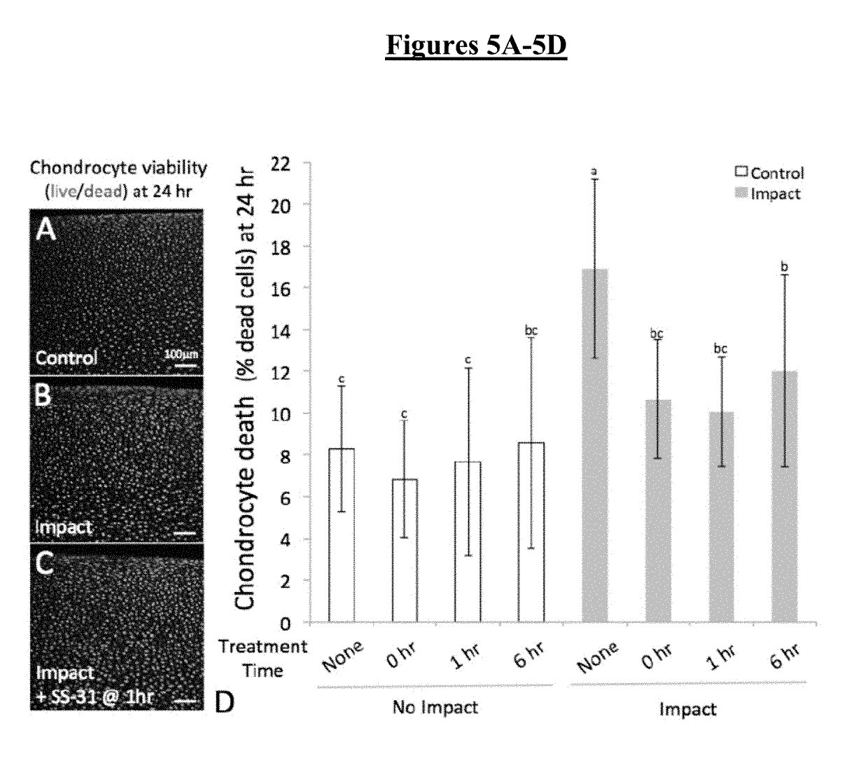

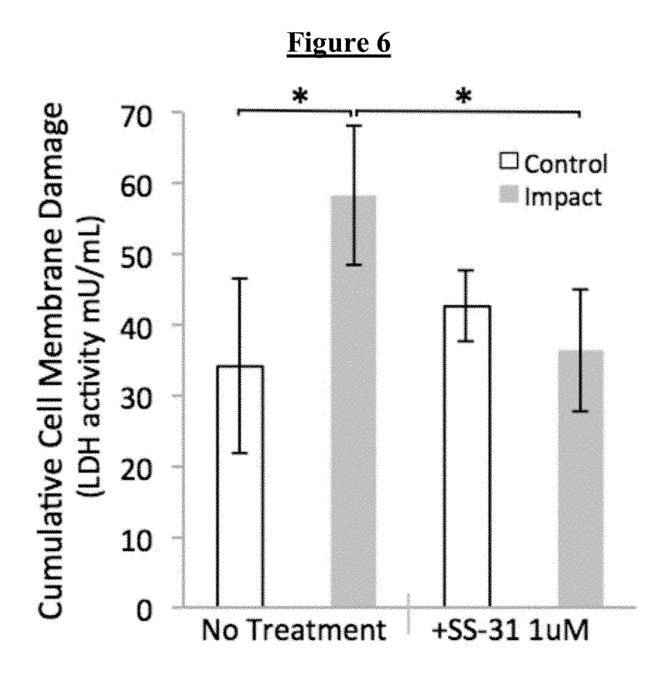

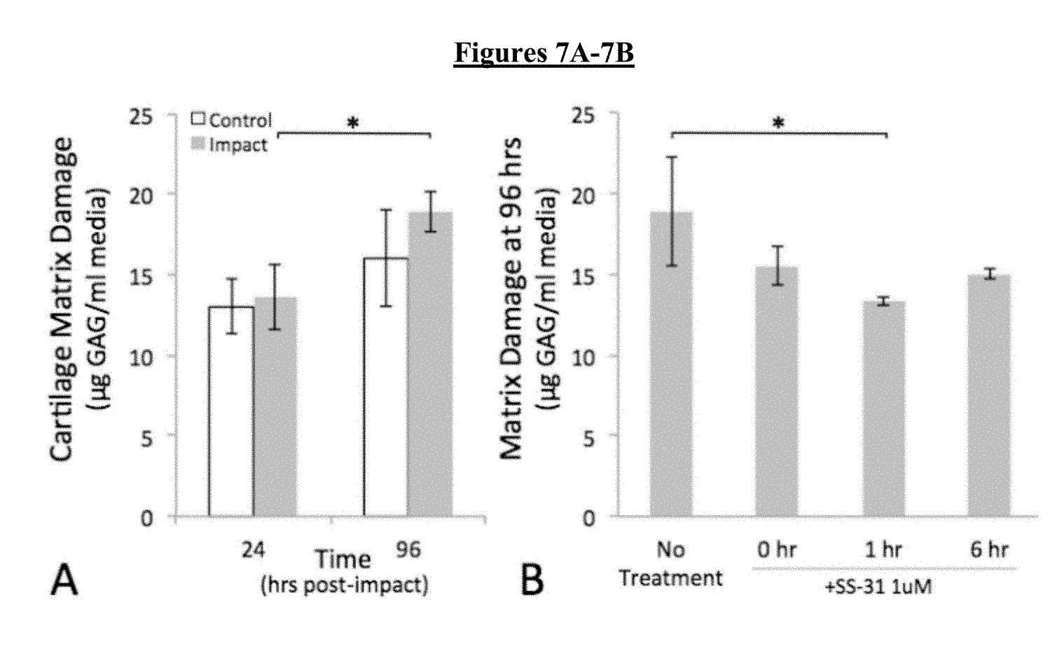

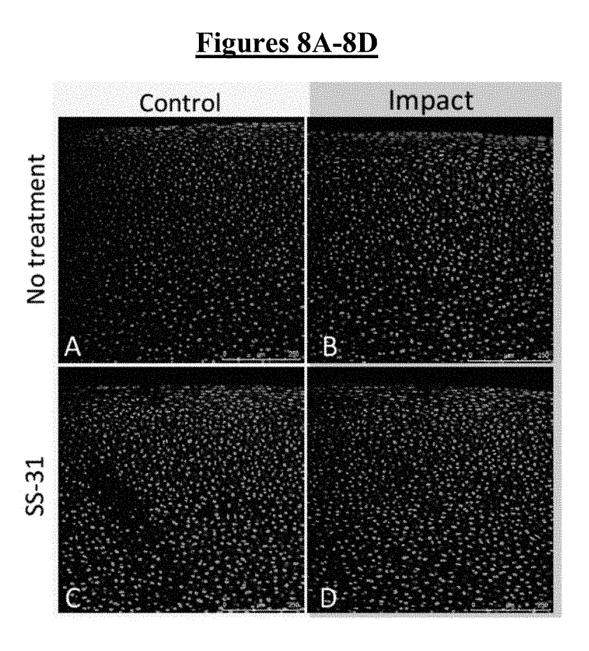

D-Arg-2′,6′-Dmt-Lys-Phe-NH₂ treatment at 0, 1 or 6 hours after impact significantly reduced chondrocyte death at 24 hours (FIGS. 5 and 8). Treatment at T₀ or T₁ resulted in chondrocyte viability similar to that of un-impacted controls. Cumulative cell membrane damage over the 7 days following injury was lower in treated than untreated cartilage (FIG. 6). GAG loss into the media was significantly elevated in impacted samples versus controls at 96 hour post impact (FIG. 7). Impact-induced GAG loss was decreased in explants treated with D-Arg-2′,6′-Dmt-Lys-Phe-NH₂ (FIG. 7).

The results show that treatment with D-Arg-2′,6′-Dmt-Lys-Phe-NH₂ reduces chondrocyte death, cell membrane damage, and cartilage matrix degradation after cartilage injury. Additionally, the results show that treatment with D-Arg-2′,6′-Dmt-Lys-Phe-NH₂ reduced mechanical injury-induced chondrocyte death, cell membrane damage, and cartilage matrix degradation even after treatment with D-Arg-2′,6′-Dmt-Lys-Phe-NH₂ was delayed by up to 6 hours after injury. Accordingly, the aromatic-cationic peptides of the present technology, or pharmaceutically acceptable salts thereof, such as acetate, tartrate salt, or trifluoroacetate salt, including, but not limited to, D-Arg-2′,6′-Dmt-Lys-Phe-NH₂, are useful in treating, preventing, or ameliorating OA or PTOA.

### Example 3

### Ex Vivo Model for Acute Impact-Induced Cartilage Injury

This example demonstrates an ex vivo model for measuring mechanical injury induced chondrocyte death and cartilage degeneration in articular cartilage.

### Methods

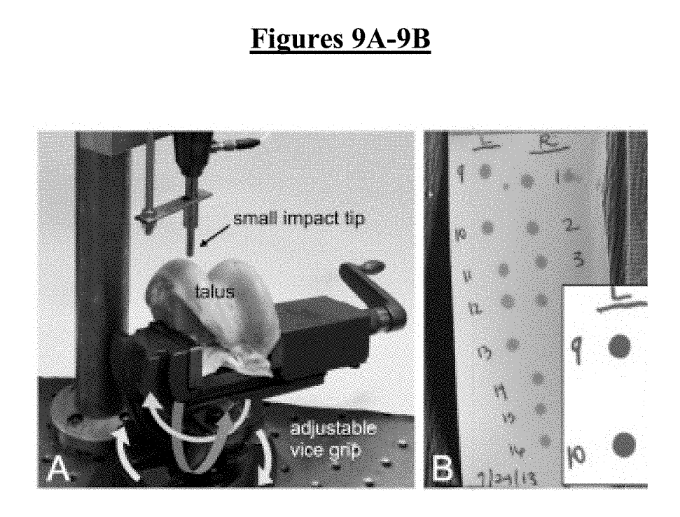

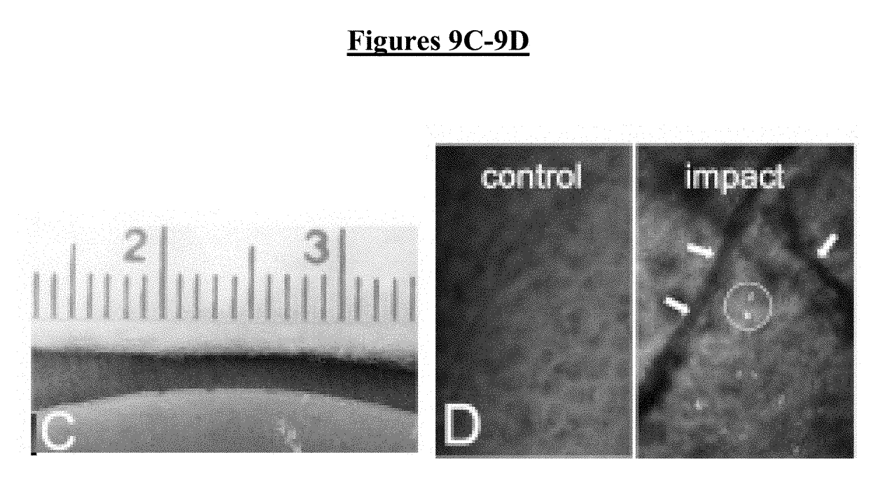

Tissue collection and handling. Osteochondral (OC) blocks comprising the medial and lateral trochlea of the right and left talus were harvested from 6 normal adult horses (ages 2-11 years) immediately following euthanasia, and incubated in phenol red-free MEM supplemented with HEPES (25 mM), penicillin (100 IU/ml), and streptomycin (100 μg/ml). OC blocks were mounted in an impact device with an adjustable vice grip. The OC blocks were positioned with the articular surface perpendicular to the direction of impact (FIG. 9A). While mounted in the device, samples were kept moist by continuous lavage with phosphate buffered saline (PBS).

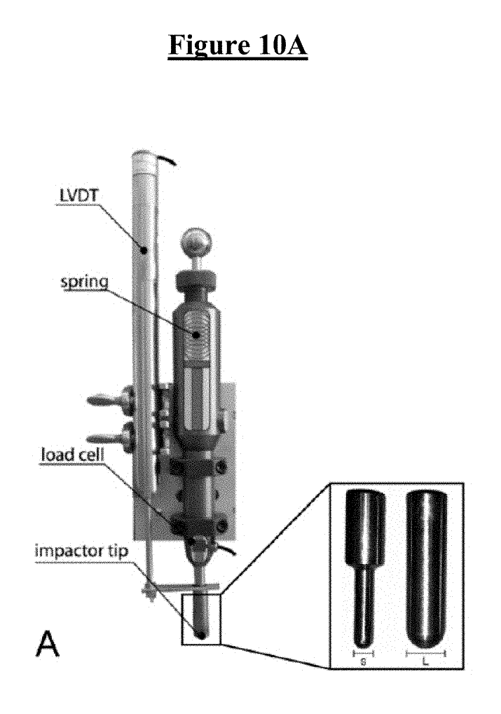

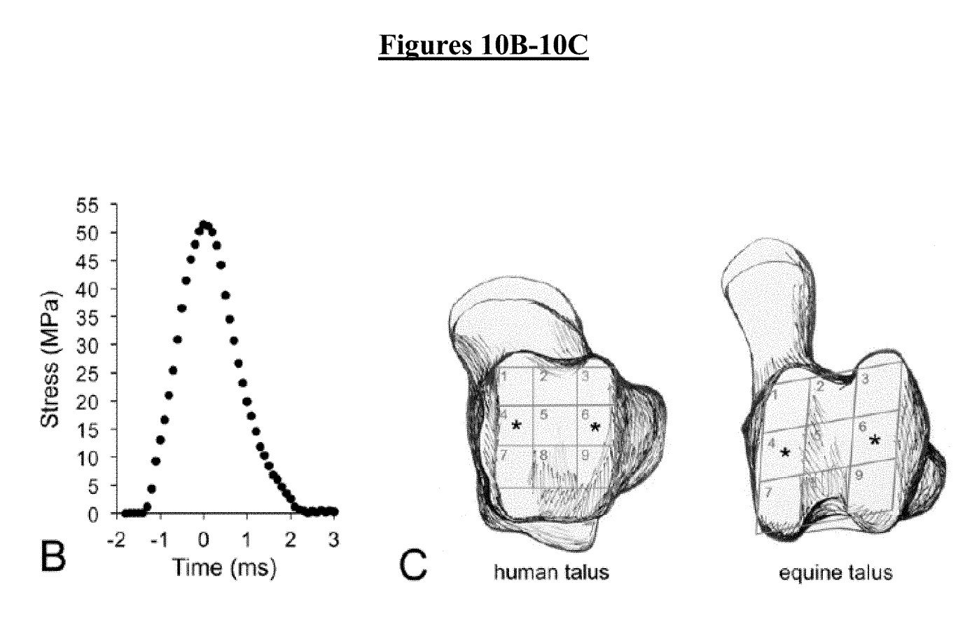

Impactor. A spring-loaded impacting device (FIG. 10A) was used to impact the OC blocks of the equine talus described above. An adjustable vice grip, capable of rotation on 3 axes, was installed below the impactor armature (FIG. 9A). One of 2 hemispherical impacting tips, differing in diameter and radius of curvature, were mounted on the end of the spring-driven missile contained within the device. A load cell (PCBPiezotronics, Depew, N.Y.) mounted in-line between the missile and the impacting tip was used to measure impact force. A linear variable displacement transducer (LVDT; RDP Electronics, Pottstown, Pa.) was attached to the impacting tip to measure displacement (FIG. 10A)

Impact and mechanical analysis. The articular surface of the talus was impacted in regions corresponding to the highest incidence of naturally occurring OC lesions in humans. A total of 180 impacts (6-10 impacts per OC block, spaced approximately 0.5 cm apart) of varying magnitudes were applied to the mid-medial and lateral trochlea of the talus using one of 2 curved impacting tips (FIGS. 10A-C). Load cell and LVDT output (voltage) were acquired simultaneously at 50 kHz with a custom LabVIEW program (NI, Austin Tex.). Cartilage thickness (t) was measured by modified needle probe technique on a mechanical testing frame (EnduraTEC ELF3200, EnduraTec, Minnetonka, Minn.) and validated by manually cutting and photographing OC blocks in cross-section adjacent to impacts, and then measuring thickness on digital images using ImageJ software (Mac OS X version 10.2, Wayen Rasband, U.S. National Institutes of Health, Bethesda, Md., USA; FIG. 9C). LVDT output was converted to displacement (d), then strain was calculated as (d)/(cartilage thickness). Load cell data (voltage) was converted to force (F), then average peak stress was calculated as (max F)/(contact area of indenter) recorded by pressure sensitive film (FujiFilm Prescale, Tokyo, Japan) and measured using ImageJ (FIG. 9B).

Multiphoton imaging and histology. Impacted OC blocks were incubated in media for approximately 2 hours, then full-thickness cartilage sections containing the impact or control site were cut off the bone and placed in 1 μM sodium fluorescein (AK-FLOUR 25%, Akorn, Inc., Lake Forest, Ill.) in PBS for 15 minutes to stain dead cells. Samples were then imaged on a multiphoton microscope using a Ti:sapphire laser at 780 nm excitation. Images were acquired at the articular surface in the transverse plane (i.e., parallel to the articular surface). Dead cells were quantified using a custom ImageJ macro and extracellular matrix (ECM) microcracks were assessed qualitatively (FIG. 9D). Impacted and control cartilage samples were fixed in 4% paraformaldehyde, then sectioned and stained with hematoxylin and eosin (H&E) and safranin O/fast green to assess structural damage, acute cellular necrosis and proteoglycan content.

### Results

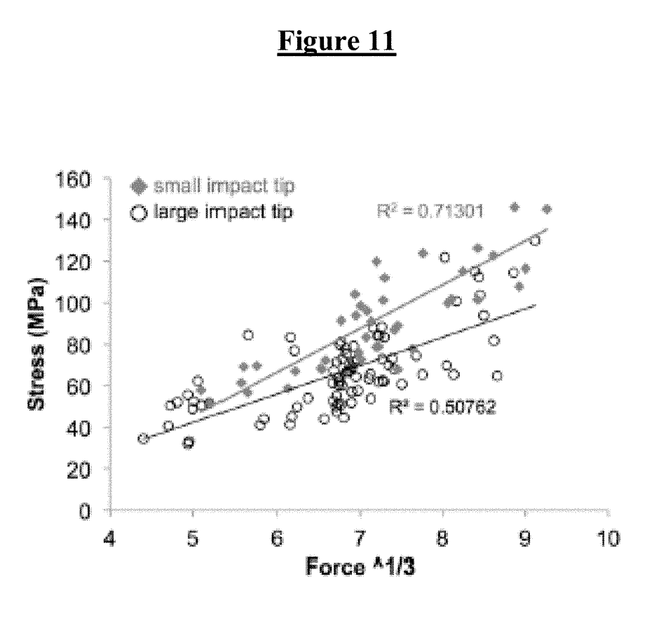

Based on Hertzian contact mechanics, the relationship between impact stress and impact force was determined. The impact stresses for cartilage under impact from a spherical tip correlated well with impact force 1/3 as predicted by Hertzian contact mechanics (FIG. 11). For both the small and large radius impacting tips, the correlations provided R²=0.71 and R²=0.50, respectively. The increased slope between the small and large radius tips is an effect of contact area, where smaller contact areas provide larger stresses at the same force level.

Joint fluid and histology results were consistent with the development of early PTOA in all injured joints. The severity of focal osteochondral injury correlated to the magnitude of impact delivered (r²=0.80, p=0.016).

These results show that impact on OC blocks comprising the medial and lateral trochlea of the right and left talus causes early PTOA. As such, mechanical injury leads to OA or PTOA.

### Example 4

### In Vivo Model for Acute Impact-Induced Cartilage Injury

This example demonstrates an in vivo model for measuring mechanical injury induced chondrocyte death and cartilage degeneration in articular cartilage.

### Methods

Animal subjects. Two healthy, young adult (3 year old) female horses were anesthetized and three impacts of varying magnitudes were applied to the left and right talus (n=4 joints) under arthroscopic guidance. Prior to surgery, all talocrural (TC) joints were deemed free of pre-existing OA by two board certified equine surgeons on the basis of normal physical examination, gait evaluation, and synovial fluid analysis. One healthy, young adult (3 year old) female horses served as the un-operated control.

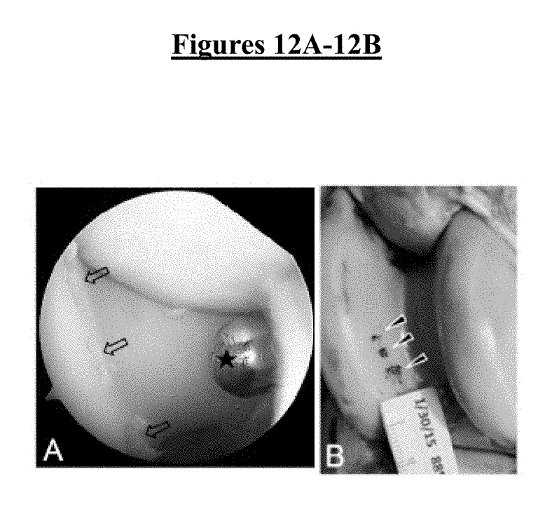

Surgical technique. An arthroscope was inserted and the TC joints were inspected for preexisting pathology. An instrument portal was created half way between the arthroscope portal and the lateral malleolus. The impactor tip was inserted into the joint and positioned perpendicular to the articular surface of the axial aspect of the medial trochlea of the talus. The impactor was held in contact with the articular surface, and the impactor trigger was depressed. The joint was then flexed several degrees, the spring tension was adjusted to set the impact magnitude, and the impactor spring was compressed. A second impact was delivered ˜5 mm distal to the first. This was repeated a third time, so that a total of 3 impacts were created along the mid-distal medial trochlea of the talus (FIGS. 12A-B). Load cell data were recorded and analyzed, as described above.

Postoperative monitoring and synovial fluid analysis. Postoperatively, horses were examined daily for clinical evidence of pain (lameness). Joint effusion was scored on a 4-point scale (Table 8). Synovial fluid was obtained weekly, starting 1 week postoperatively for 4 weeks, then at 6, 8, and 12 weeks postoperatively. Synovial fluid cytology was evaluated by a board certified veterinary clinical pathologist, and total protein, nucleated cell count, and differential cell counts were measured. Synovial fluid characteristics (viscosity, color, turbidity) reported by the clinical pathologist were combined into a single joint inflammation score (Table 7). Additional aliquots of synovial fluid were stored at −80° C. until further analysis, when synovial fluid biomarkers of early OA (PGE2 and TNFα) were measured on ELISA. Cytokine concentration was quantified using commercial ELISA kits (PGE2 ELISA kit, Enzo Life Sciences catalog #ADI-900-001 and Equine TNF alpha ELISA Kit, Thermo Scientific ESS0017). The assays were completed using undiluted synovial fluid according to manufacturers' directions, and the 96-well plate was read on a spectrophotometric microplate reader (Tecan Safire; Männedorf, Switzerland).

TABLE 7
Rubric for joint inflammation score

| Analysis | Parameter | Score | Qualifications |
|---|---|---|---|
| Synovial Fluid | Color/clarity/viscosity | 0 | Normal |
| | | 1 | Abnormal |
| | Total Protein | 0 | <2.5 |
| | | 1 | 2.5-4 |
| | | 2 | >4 |
| | Nucleated cell count | 0 | <1000 |
| | | 1 | 1000-9000 |
| | | 2 | >9000 |
| | % Neutrophils on | 0 | 0-15% |
| | differential cell count | 1 | 15-65% |
| | | 2 | >65% |
| | Cellular morphology | 0 | Normal |
| | | 1 | Abnormal |
| Clinical | Joint effusion | 0 | None (Normal) |
| Examination | | 1 | Mild |
| | | 2 | Moderate |
| | | 3 | Severe |

Tissue collection, gross pathology and histopathology. Horses were sacrificed 6 or 12 weeks postoperatively to examine acute stages of PTOA. India ink was applied to the articular surface of the medial talus to identify areas of cartilage cracking and fibrillation. Suspect areas of impact (well-circumscribed, circular areas of intense India ink uptake on the axial aspect of the mid-distal aspect of the medial trochlea of the talus) were identified, and this information was cross-referenced with arthroscopic videos obtained during surgery to confirm the location of individual impact sites (FIGS. 12A-B). OC blocks containing each of the three impact sites, the opposing articular surface (distal intermediate ridge of the tibia; DIRT) and the un-injured lateral trochlear ridge of the talus (LTR) were harvested, fixed in 4% paraformaldehyde, and decalcified using 20% sodium citrate and 44% formic acid. OC sections were stained with H&E and Safranin O/Fast green. Histology was scored by two independent observers (MLD, LD) blinded to treatment (i.e., injury status and magnitude), based on a 24-point modified OARSI scoring system (Table 8) and using the 6-point OARSI grading system. Synovial membrane was harvested, processed and stained with H&E and consensus scored (Table 9) by one experienced observer (MLD) and a board certified clinical pathologist (ADM) blinded to treatment.

TABLE 8
Modified OARSI osteochondral histology scoring
for in vivo impact model

| Analysis | Score | Qualifications |
|---|---|---|
| Cartilage Structure | 0 | None (Normal) |
| (Fibrillation/fissuring of the | 1 | Restricted to surface/superficial |
| articular cartilage surface) | | zone |
| | 2 | Fissures/clefts extends into middle |
| | | zone |
| | 3 | Extends to level of deep zone |
| | 4 | Extends into the deep zone |
| | 5 | Full thickness loss (to calcified |
| | | cartilage) |
| Tidemark/subchondral bone | 0 | None (Normal) |
| remodeling | 1 | Duplication of tidemark, |
| | | advancement of SC |
| | | bone into calcified cartilage, |
| | | scalloped margins |
| | 2 | Advancement of SC bone through |
| | | tidemark(s) |
| | 3 | Complete disruption/ |
| | | disorganization of tidemark, |
| | | SC bone |
| Chondrocyte necrosis | 0 | Normal |
| (Necrotic cells near the | 1 | 1 necrotic cell |
| articular surface per 20X | 2 | 1-2 necrotic cells |
| objective) | 3 | 2-3 necrotic cells |
| | 4 | 3-4 necrotic cells |
| Focal cell loss | 0 | Normal |
| (Area of acellularity per 20x | 1 | 10-20% |
| field) | 2 | 20-30% |
| | 3 | 40-50% |
| | 4 | >50% |
| Cluster (complex chondrone) | 0 | None |
| formation | 1 | 2 chondrocytes |
| | 2 | 2-3 chondrocytes |
| | 3 | 3-4 chondrocytes |
| | 4 | >4 chondrocytes |
| Loss of GAG staining | 0 | Normal |
| (on SOFG) | 1 | <25% loss |
| | 2 | 25-50% |
| | 3 | 50-75% |
| | 4 | >75% |

TABLE 9
Synovial membrane histopathology scoring for in vivo impact model

| Analysis | Score | Qualifications |
|---|---|---|
| Inflammatory cell | 0 | None |
| infiltration | 1 | Mild presence in 25% |
| | 2 | Moderate presence in 25-50% |
| | 3 | Marked presence in >50% |
| Vascularity | 0 | Normal |
| (Number of vessels) | 1 | Mild increase in focal areas |
| | 2 | Moderate increase up to 50% |
| | 3 | Marked increase in >50% |
| Intimal hyperplasia | 0 | None |
| | 1 | Villi with 2-4 rows intimal cells |
| | 2 | Villi with 4-5 row |
| | 3 | Villi with >5 rows |
| Subintimal edema | 0 | None |
| | 1 | Mild edema in 25% |
| | 2 | Moderate edema in 25-50% |
| | 3 | Marked edema in >50% |
| Subintimal fibrosis | 0 | Normal |
| | 1 | Mild increase in 25% |
| | 2 | Moderate increase in 25-50% |
| | 3 | Marked increase in >50% |

Statistical analysis. The relationship between impact force and impact stress was determined based on Hertz's contact theory where stress is related to force1/3. Linear regression was conducted between these 2 variables. The relationship between OARSI score at 12 weeks post-impact and peak impact stress was determined using linear regression.

### Results

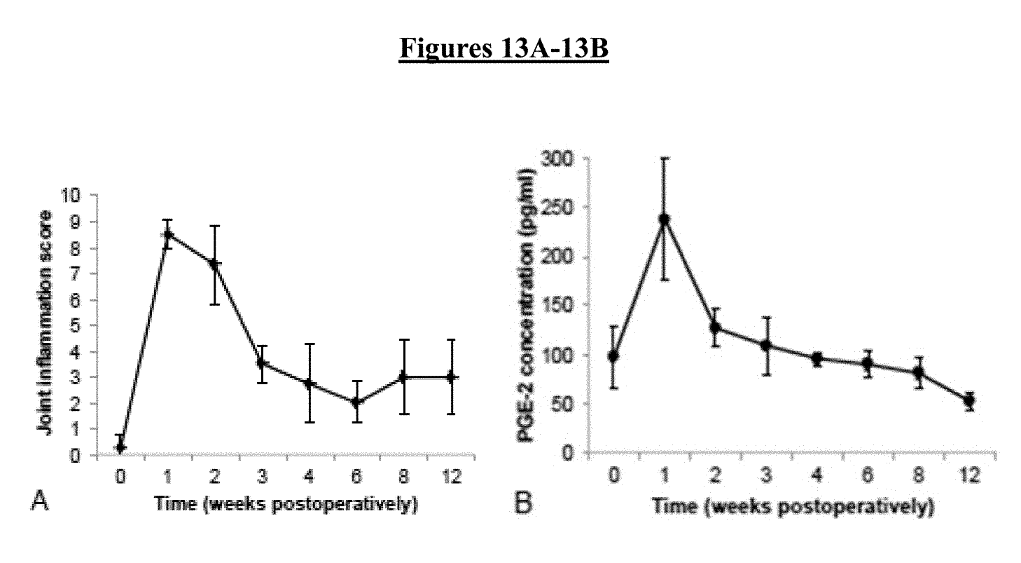

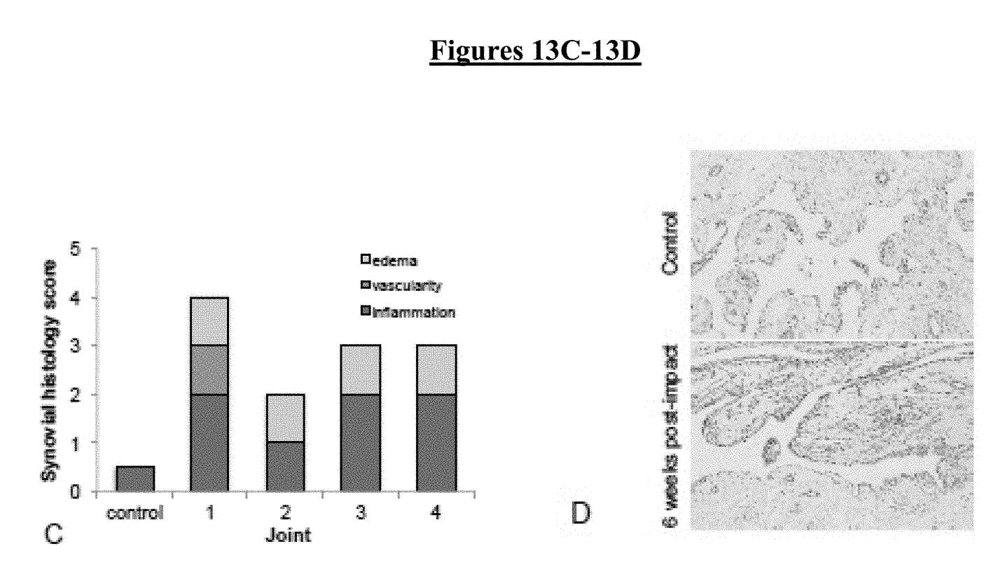

Clinical observations and synovial fluid analysis. No major complications were experienced intra- or post-operatively. Mild to moderate synovial effusion was present in all impacted joints and decreased gradually throughout the study period, with no observable lameness at any time point. Based on synovial fluid analysis and clinical observation, joint inflammation was reduced after 2 weeks of impact; however, joint inflammation scores did not return to baseline and remained elevated throughout the 12-week study (FIG. 13A), which indicates low-grade pathology throughout the course of the study. PGE-2 increased an average of 2.5 fold (range 1.5-4×) 1 week postoperatively, and returned to baseline by 4 weeks in 3 of the 4 joints (FIG. 13B).

Synovial membrane histopathology. Histopathologic examination of the synovial membrane from injured joints indicated mild to moderate inflammation, with or without subintimal edema and/or increased vascularity (FIGS. 13C-D). None of the synovium sections showed evidence of subintimal fibrosis or intimal hyperplasia. These changes indicate mild to moderate synovitis at 6 and 12 weeks post-injury, in agreement with synovial fluid analysis results.

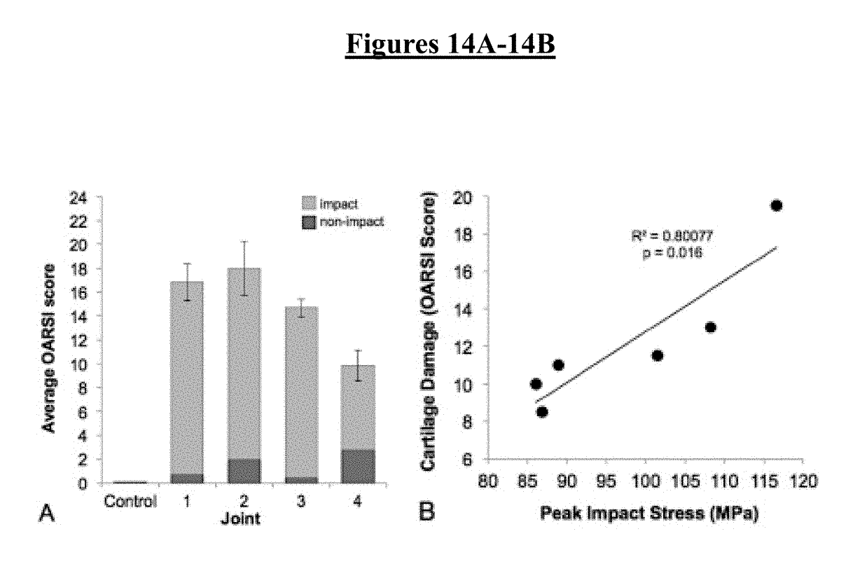

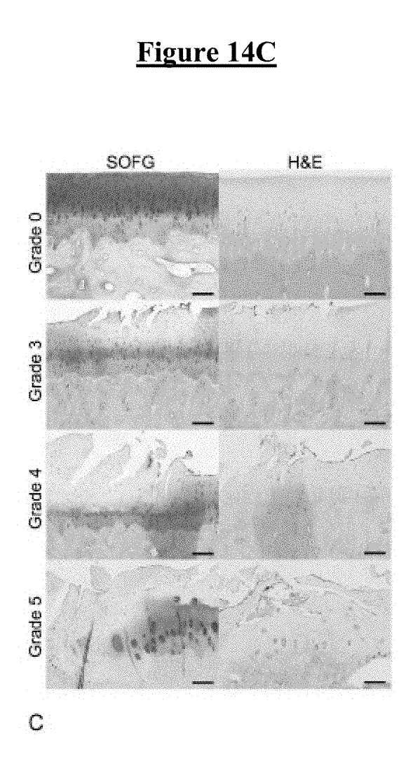

Gross and histopathologic osteochondral lesions. At necropsy, all impact sites were grossly identified with the application of India ink. Typically, impacts were easily distinguishable as a cluster of radiating cracks, and in all cases correlated well to images obtained at arthroscopy (FIG. 12B). All OC sections from areas of impact showed histopathological evidence of early OA-type lesions (FIGS. 14A-C). Changes ranged from mild to severe erosions, cracking/fissuring, hypocellularity, chondrocyte necrosis and clonal expansion of chondrocytes with an average modified OARSI score of 14.8 (s.d. 4.0) out of 24 and a mean OARSI Grade 19 of 3.5 (s.d. 1.1) out of 6 (FIGS. 14A-C). At 3 months following injury, OARSI grade for individual impacts correlated with impact magnitude (r=0.8953, p=0.016; FIGS. 14A-C).

These results show that impact on TC joints causes early OA-like osteochondral lesions and impact stress cartilage damage. As such, mechanical injury leads to OA or PTOA.

### Example 5

### Acute Mitochondrial Dysfunction in Cartilage Following Mechanical Injury

This example demonstrates that mitochondrial dysfunction is an acute response of chondrocytes to mechanical injury.

### Methods

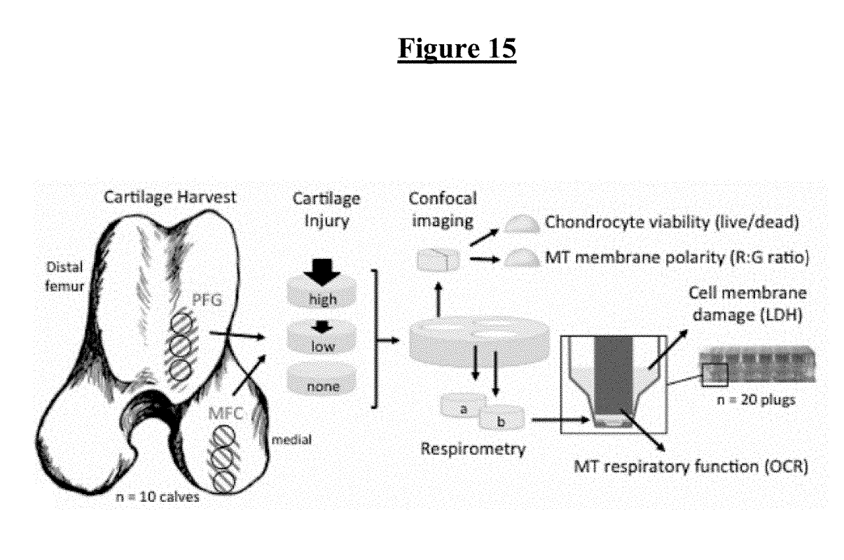

Cartilage Harvest and Mechanical Injury. Healthy bovids (n=10; 1-3 days of age) were obtained from a livestock auction and humanely euthanized in accordance with AVMA guidelines. Within 12 hours of sacrifice, full thickness cartilage explants were harvested from both the left and right knee joints, using an 8 mm biopsy punch (FIG. 15). Explants were rinsed in phosphate-buffered saline (PBS), trimmed to a uniform thickness of 3 mm from the articular surface using a custom jig, and placed in cartilage explant media (phenol-free DMEM containing FBS 10%, HEPES 0.025 ml/ml, penicillin 100 U/mL and streptomycin 100 U/mL).

Explants were subjected to a single, rapid impact injury using a validated model (see Bonnevie et al., Cartilage, 6(4):226-232 (2015)) or served as un-injured controls. Explants were positioned in a well containing media under the plane-ended tip of a spring-loaded impacting device. Impact magnitude was adjusted by setting the deflection of the impactor's internal spring. During impact, force was measured at 50 kHz by an in-line load cell (PCB Piezotronics, Depew, N.Y.) and displacement was measured by a linear variable differential transducer (LVDT; RDP Electronics, Pottstown Pa.) attached to the impactor tip. Voltages from the load cell and LVDT were recorded simultaneously with a custom LabVIEW program (NI, Austin Tex.) and mechanical parameters for each impact were calculated as described in Bonnevie et al.

Characterization of In Situ Chondrocyte Mitochondrial Respiratory Function Immediately Following Cartilage Injury. Real-time microscale respirometry was used to measure chondrocyte mitochondrial respiratory function in explanted cartilage. Explants (n=65 total) from the medial femoral condyle (MFC) were harvested and impacted over a broad range of injury magnitudes (M1-M4; 5-17 MPa, 5-34 GPa/sec). This range was selected based on preliminary trials (160 explants, 8 trials) and previous work to determine the stress and stress rate thresholds associated with cell death and extracellular matrix damage in this system. The goal was to apply a range of injury magnitudes, from minimal cell death (M1) to cell death without surface cracking (M2) to subcritial damage (i.e., impacts that produced surface fissuring but not full thickness defects; M3 and M4, Table 10).

TABLE 10
Mechanical parameters of impact by experimental group

Impact Magnitude

| Experimental | Mean Peak | Mean Peak Stress Rate; GPa/sec |
|---|---|---|
| Group | Stress; MPa | (+/−s.d.) |
| Control | n/a | n/a |
| M1 | 5.6 (0.4) | 6.7 (1.3) |
| M2 | 7.5 (0.4) | 9.3 (1.5) |
| M3 | 14.1 (0.7) | 28.1 (1.8) |
| M4 | 16.2 (0.7) | 32.0 (1.6) |

Following impact, two cartilage disks (3 mm diameter×500 μm thickness from the articular surface) were prepared and immediately loaded into a randomly assigned well of a 24-well tissue capture microplate (Seahorse Biosciences, Billerica, Mass.) containing assay media (bicarbonate-free DMEM supplemented with 2.5 mM glucose, 2 mM L-glutamine, 2 mM pyruvate, and 1% FBS). Following a calibration cycle, glycolysis and oxidative phosphorylation were quantified every 8 minutes for a minimum of 225 minutes by measuring extracellular acidification (ECAR) and oxygen consumption rates (OCR) within each well, respectively using an XF24 Extracellular Flux Analyzer (Seahorse Biosciences). After basal respiration was measured for at least 40 minutes, a mitochondria stress test was performed according to standard protocols. Briefly, OCR was measured in response to the automated sequential addition of: 1) oligomycin (1.5 μM), an ATP synthase inhibitor; 2) carbonyl cyanide-4-(trifluoromethoxy) phenylhydrazone (FCCP; 1.0 μM), a proton circuit uncoupler; and 3) a combination of rotenone (0.5 μM)+antimycin A (1.0 μM), inhibitors of mitochondria complexes I and III, respectively (Seahorse Biosciences). The remainder of each explant was used to determine chondrocyte density and viability, as described below, in order to normalize respirometry data to viable cell number on an individual explant basis. Data were normalized to viable cell number by dividing OCRs measured in each well containing a single cartilage plug, by the number of viable cells in that well. Mitochondria functional indices were calculated as described in Brand et al., Biochem J., 435(2):297-312 (2011) using OCR values as follows: basal OCR (bOCR)=initial OCR−non-mitochondrial respiration (NMR); maximal (uncoupled) respiration (mOCR)=FCCP stimulated OCR−NMR; spare respiratory capacity (SCR)=(uncoupled respiration−NMR)−(bOCR−NMR); Proton leak=(oOCR−NMR).

Chondrocyte Viability and Cell Membrane Damage Assays. In order to determine cell density and quantify chondrocyte viability, cartilage was placed in PBS containing calcein AM (2 μM) and ethidium homodimer (1μM) for 30 minutes at 37° C. in the dark, to stain live and dead cells, respectively. Explants were then rinsed in PBS and imaged on a Leika SP5 confocal microscope. Digital z-stacked images were acquired in two channel sequential scans (488/498-544 and 514/563-663 nm excitation/emission, respectively) using a modified 3D scanning protocol consisting of 10 z-stacked 512×512 pixel (387.5 μm×387.5 μm) images spaced 10 μm apart in the z plane at 20× magnification. The number of live, dead, and total cells in each image was quantified using a custom ImageJ macro. The explant volume and chondrocyte density were calculated for each explant and used to normalize respirometry data to viable cell number.

As a measure of cell membrane damage, lactate dehydrogenase (LDH) activity was assayed in cartilage-conditioned media from each well of the XF assay plate, according to manufacturer's instructions (Sigma-Aldrich, St. Louis, Mo.). Briefly, equal volumes of cartilage-conditioned media and kit reagent were added to a 96-well plate and absorbance was measured at 450 nm in 5-minute intervals by a spectrophotometric microplate reader (Tecan Safire; Mannedorf, Switzerland). To establish a post-impact LDH release time-course and validate the use of media obtained from the XF assay plates following microrespirometry assays, cartilage explants (n=16) were impacted at the magnitudes described above (M1-M4: Table 11), and incubated for 24 hours in cartilage explant media. LDH assay was performed on cartilageconditioned media at 1, 5, 7 and 24 hours after impact.

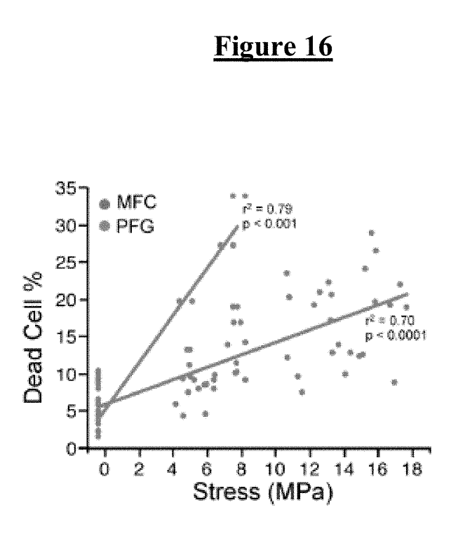

Comparison of chondrocyte response to injury between two locations within the same joint. Explants were harvested from the medial femoral condyle (MFC) and distal patellofemoral groove (PFG) (n=40 total). The MFC is the main weight-bearing surface of the knee, while the distal PFG is a non-weight bearing articular surface. Three explants were harvested from two locations within each joint. One explant from each area was subjected to one of 3 impact treatments; lower magnitude (M1; 5.6±0.4 MPa mean peak stress, 6.7±1.3 GPa/sec mean peak stress rate), higher magnitude impact (M2; 7.5±0.4 MPa, 9.3±1.5 GPa/sec) or non-impacted control. Microrespirometry was performed (n=8/group) and data was normalized. Impact magnitudes (M1 and M2) were chosen based on preliminary data, which revealed that impact above ˜8 MPa peak stress (˜11GPa/sec peak stress rate) in the PFG resulted in extensive cell death, preventing comparisons to the MFC (FIG. 16).

Mitochondrial Membrane Polarity Assay. The functional integrity of the inner mitochondrial membrane was assessed in situ using confocal imaging of fluorescent mitochondria probes. Following impact and sectioning, samples (n=40) were placed in PBS containing tetramethylrhodamine methyl ester perchlorate (TMRM;10 nM, Molecular Probes), MitoTracker Green (MTrG; 200 nM, Molecular Probes, Eugene, Oreg.), and Hoechst 33342 (1 μg/ml, Molecular Probes) for 40 minutes and protected from light. TMRM is a polarity-sensitive mitochondrial probe, and red fluorescence indicates active transport of the dye across a polarized (functional) mitochondria membrane. MTrG is a polarity-insensitive mitochondrial probe, which stains all mitochondria regardless of mitochondria membrane potential. Hoechst acts as a nuclear counterstain, and preferentially stains cells with compromised plasma membranes. After staining, explants were rinsed in PBS and imaged on a Leica SP5 confocal microscope. Images were acquired and analyzed (for live/dead staining), with the exception that 1024×1024 pixel (775 μm×775 μm) images were acquired in three channel sequential scans (405/411-497, 488/498-544 and 561/569-611 nm excitation/emission, respectively) spaced 5 μm in the z-plane at 20× magnification and red:green florescent intensity (R:G) ratios for each image were determined using a custom ImageJ macro. Macros for each imaging channel were optimized and image-wide analysis of R:G ratio was validated by manual ROI selection of individual cells at higher (40×) magnification for control and impacted explants.

Statistical Analysis. The response variables bOCR and mOCR were analyzed using a linear mixed effects model with fixed effects of treatment group and site (MFC or PFG) and random effect of trial (animal) and limb (left or right). The unit of study was a cartilage explant. The relationship between impact magnitude (stress and stress rate) and chondrocyte death was analyzed using a linear regression model. A one-way ANOVA was used to compare response variables between treatment groups. Post-hoc pairwise comparisons between treatment groups were performed using Tukey's HSD method to control for multiple comparisons. Residual analyses were performed on log-transformed data to ensure the assumptions of normality and homogeneous variance were met. Differences were considered statistically significant when p<0.05. All statistical analyses were performed using JMP Pro Version 11.0 (SAS Inc.) software.

Microscale Respirometry. Each well of a specialized XF24 islet capture microplate (Seahorse Biosciences) was preloaded with assay media (bicarbonate-free DMEM supplemented with 2.5 mM glucose, 2 mM L-glutamine, 2 mM pyruvate, and 1% FBS). The XF sensor cartridge is equipped with 4 injection ports per well, which allow automated addition of drugs during the experiment. Approximately one hour prior to experimentation, three of the four injection ports were preloaded with mitochondria inhibitors to perform a mitochondria stress test. Concentrations for mitochondria inhibitors were determined by preliminary dose response optimization assays. The sensor cartridge was allowed to equilibrate in a 0%-CO₂ incubator prior to being loaded into the XF24 analyzer for calibration.

Cartilage slices were prepared as follows: immediately following cartilage injury, a 3 mm biopsy punch was used to harvest 2 cylindrical plugs from each explant. The plugs were then trimmed to 500 μm thickness from the articular surface using a custom jig cartilage using a custom cutting jig and tissue slicer blade (Thomas Scientific, Swedesboro, N.J.). Cartilage was kept hydrated, each cut was performed with a new and lubricated instrument, and handling of the tissue was strictly minimized. Cartilage slices (n=20 per assay; 3 mm diameter by 500 μm thick) were loaded into the islet capture plate, articular surface facing up, then the capture screens were snapped in place to retain the cartilage at the bottom of each well. Four wells containing media only served as background control wells. The plate was equilibrated in a 0%-CO₂ incubator for one hour and then loaded into a Seahorse XF24 analyzer for analysis. The time between cartilage impact and start of the assay was a mean of 154 minutes (range 143-167).

Image analysis. Digital image analysis was carried out using ImageJ software (Mac OS X version 10.2, Wayen Rasband, U.S. National Institutes of Health, Bethesda, Md., USA) with macros customized for each imaging channel. Key parameters including pixel intensity threshold and max/min particle size were optimized based on manual counts of a minimum of 5 z-stacks obtained from both control and impacted explants. Following optimization, all images were digitally analyzed using the same macro as follows: 1) Each individual image in a stack was thresholded based on mean pixel intensity of that image and 2) then individual particles were identified and counted based on particle size. A mean value (e.g., number of dead cells) for each stack was calculated by averaging the values for all 10 images in that stack and excluding any image outside one standard deviation of the mean. At least two stacks were acquired per explant (mean 3, range 2-4) and final reported values are the mean for all stacks acquired of that explant.

Normalization of respirometry data. Calculation of cell density: For each image, the imaging field was set at 512pixels (775 μm) wide, and the depth of imaged tissue from articular surface to the bottom of the imaging field was measured digitally using the ImageJ software measuring tool, with the average depth of 700(±22.8) μm. The volume of tissue imaged was calculated as the width×average depth×10 μm slices. This resulted in an average calculated chondrocyte density of 0.22×10⁶ cells/mm³.

Calculation of explant volume: Following completion of the XF assay, explant discs were bisected, placed cut-surface down on a glass cover slip and imaged using an inverted light microscope. Digital images were obtained and the volume of each explant was calculated using ImageJ software by 2 methods: 1) the diameter of each explant was measured using the line measuring tool, 6-8 thickness (height) measurements were obtained at 90° to the diameter measurement, then the volume of the cylinder was calculated as=π(diameter/2)²×mean thickness and 2) cross-sectional area of each hemi-cylinder was measured using the tracing tool, then the volume of the explant was calculated as=π*radius² (area of cut surface/diameter). Values for individual explant volume were obtained by averaging the tissue volume obtained using both methods of calculation.

### Results

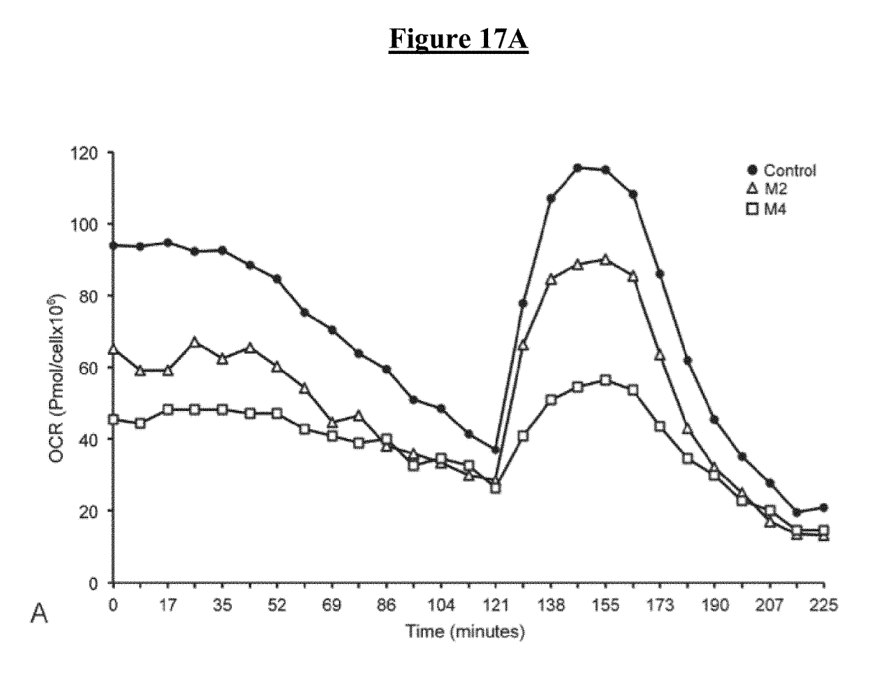

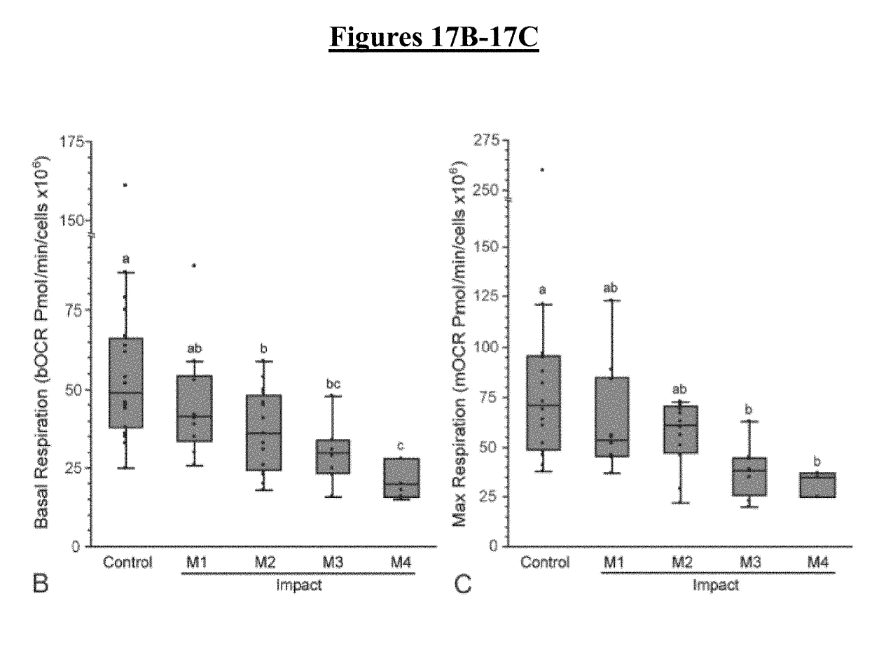

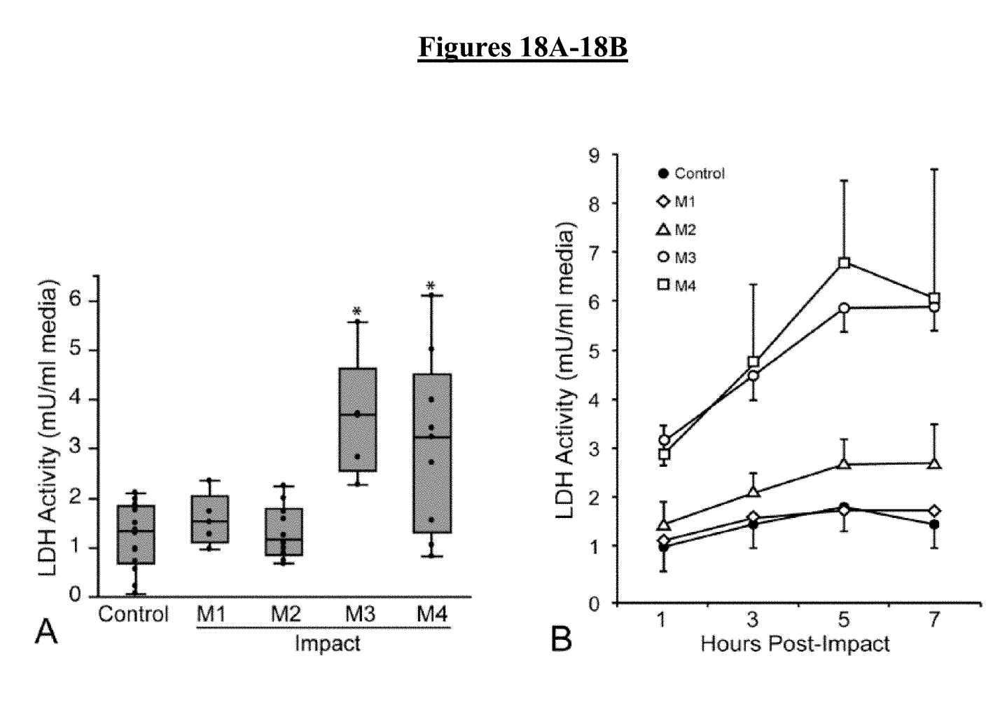

Chondrocyte respiration after cartilage injury. Mitochondrial respiratory function in MFC cartilage was assessed by measuring oxygen consumption rate (OCR) in the acute phase (from 2-6 hours) after injury. Representative curves for OCR are shown in FIG. 18A, and demonstrate differences in respiratory function between low impacted, high impacted and control cartilage. Mitochondria respiration declines with increasing injury magnitude, revealing acute impact-induced mitochondria dysfunction (FIGS. 17A-C). There was a significant effect of treatment (impact) group on bOCR (F4,56=5.4135, p=0.0009) and mOCR (F4,56=3.0572, p=0.026). Cartilage injury resulted in a 20-32% decrease in bOCR in explants from impact groups M2-M4 (FIG. 17B), and a 26-44% decrease in mOCR in groups M3 and M4 compared to un-impacted controls (FIG. 17C). Parameters of respiratory control calculated using oligomycin-inhibited OCR (oOCR) could not be reliably determined because steady state OCR following oligomycin treatment was not reached in the majority of samples (FIG. 17A). Injury had no effect on ECAR (p=0.66).

Chondrocyte death in MFC cartilage was positively correlated with impact magnitude (FIG. 16), with the strongest correlation associated with peak impact stress (r²=0.70, p<0.0001). A significant increase in cell death was observed above 7 MPa peak stress, establishing a threshold for acute chondrocyte death in this model system. Cell membrane damage was assessed in cartilage-conditioned media obtained from wells following the respirometry assay, and revealed 2-3 fold increase in LDH activity for explants impacted at higher peak stresses (M3, M4) compared to lower impacts (M1, M2) and controls (FIG. 18A). Based on the time-course experiment, LDH activity peaked at approximately 5 hours following cartilage injury at all impact magnitudes (FIG. 18B).

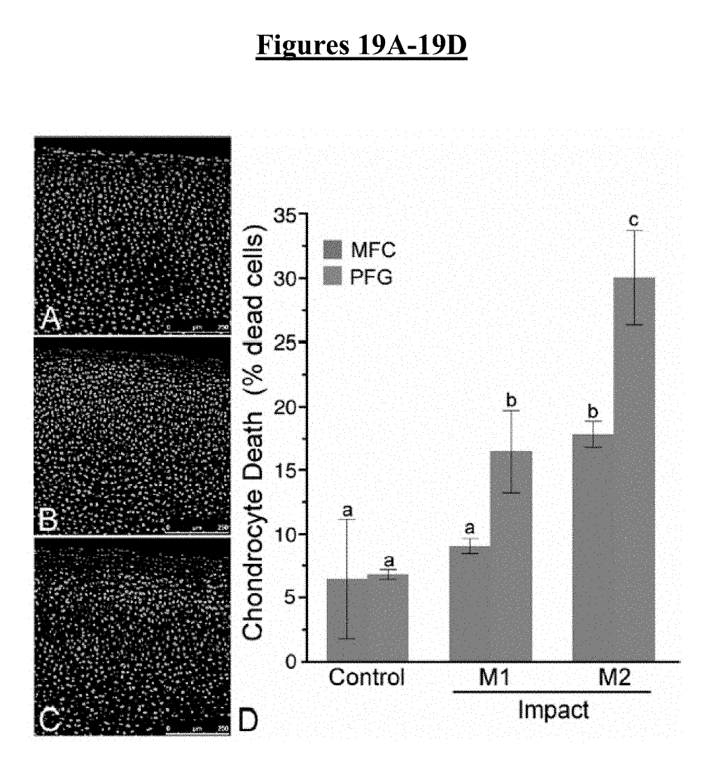

Comparison of Chondrocyte Response to Injury in MFC versus PFG cartilage. Similar to MFC explants, chondrocyte death in PFG explants was positively correlated with peak impact stress (r²=0.79, p<0.001; FIG. 16). However, chondrocytes from PFG cartilage, a non-weight-bearing articular surface, were more sensitive than the MFC to impact induced cell death. PFG explants experienced an approximately 2-fold and 5-fold increase in cell death over controls at the lower (M1) and higher (M2) impact magnitudes, respectively (FIGS. 19A-D). At lower impact magnitudes (M1), MFC viability was not affected.

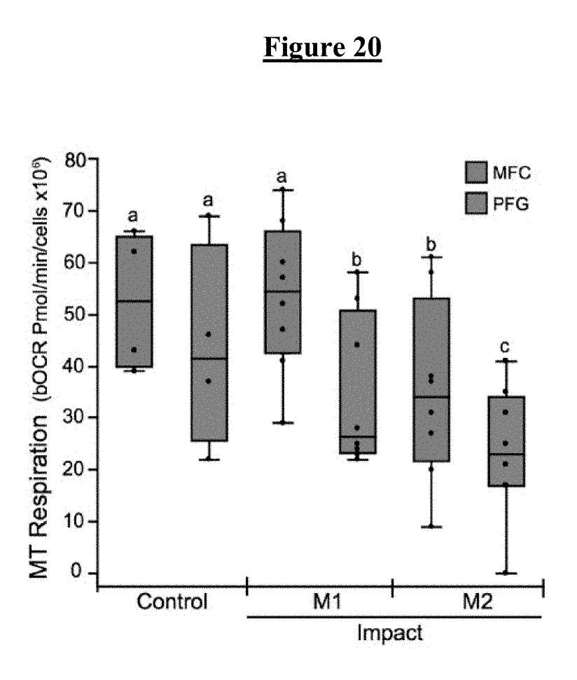

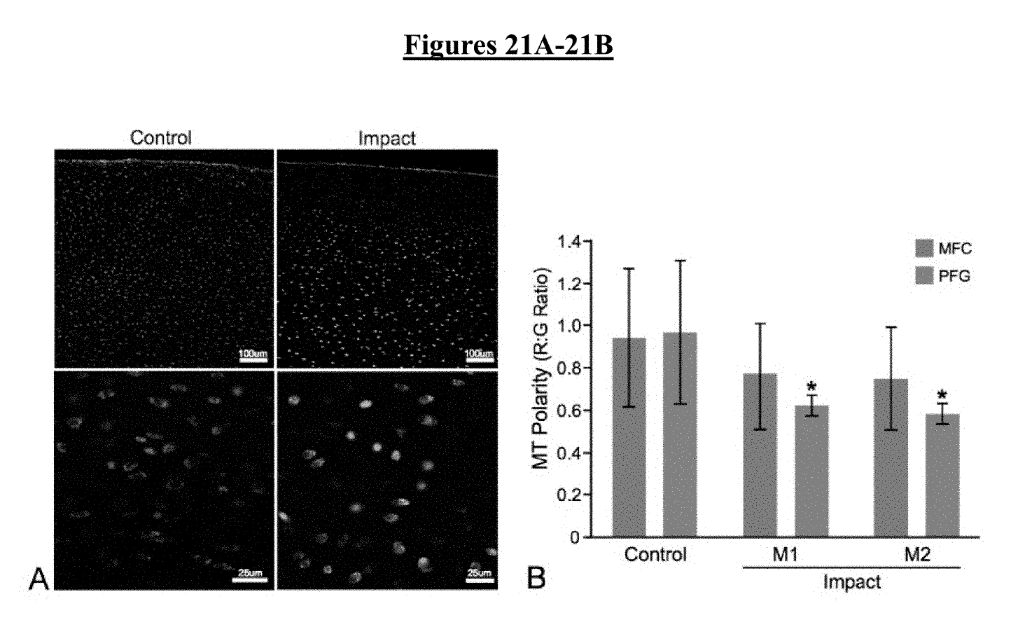

Cartilage from the PFG was more sensitive to impact-induced mitochondria respiratory dysfunction (FIG. 20). The basal oxygen consumption rate of viable PFG chondrocytes was significantly lower in groups impacted at the lowest (M1) and higher (M2) magnitudes compared to un-injured control cartilage, whereas in MFC cartilage, bOCR was only affected at the higher impact magnitude (M2). Relative mitochondria membrane potential (mitochondria polarity) was used to assess the functional integrity of the inner mitochondria membrane by calculating the R:G ratio, which represents the ratio of polarized to depolarized mitochondria within each explant. In uninjured controls, mitochondria polarity was similar in MFC and PFG cartilage. mitochondria polarity was significantly decreased in both the lower (M1) and higher (M2) impacted explants from the PFG. Over this range of impact magnitudes, no statistically significant differences were detected between control and impacted samples from the MFC (FIGS. 21A-B).

These results show that mitochondrial dysfunction is an acute response of chondrocytes to cartilage impact. The results also show that cartilage from the weight-bearing surface of the distal femur (MFC) was more resistant to impact-induced mitochondrial dysfunction and cell death than that of a non-weight bearing surface (PFG). These results also show that there are regional differences between weight bearing and non-weight bearing articular surfaces, either due to structural differences of the ECM, cellular response to injury, and/or differences in mechanotransduction.

### Example 6

### Treatment with D-Ar₂-2′,6′-Dmt-Lys-Phe-NH₂ Reduces Chondrocyte Death and Cartilage Degeneration Following Mechanical Injury

This example demonstrates that treatment with D-Arg-2′,6′-Dmt-Lys-Phe-NH₂ reduces chondrocyte death and cartilage degeneration following mechanical injury.

### Methods

Tissue harvest. Full thickness cartilage explants were harvested from the knee joints of healthy bovids (n=8 animals, 1-3 days of age) within 48 hours of sacrifice using an 8 mm biopsy punch. Specimens were rinsed in phosphate-buffered saline (PBS), trimmed to a uniform thickness of 3 mm from the articular surface using a custom jig, and placed in cartilage explant media (phenol free DMEM containing 1% FBS, HEPES 0.025 ml/ml, penicillin 100 U/mL, streptomycin 100 U/mL and 0.1 g/L glucose).

Rapid impact injury model. Explants were subjected to injury using rapid-impact model or served as unimpacted controls (as described in Example 3). Briefly, explants were positioned in a well containing PBS under the plane-ended tip of a spring-loaded impacting device. The impactor was used to deliver a single, rapid cycle of unconfined axial compression (24.0±1.4 MPa peak stress; 53.8±5.3 GPa/s peak stress rate). Impact force was measured at 50 kHz by a load cell (PCBPiezotronics, Depew, N.Y.) attached to the impactor tip. Voltage from the load cell was recorded with a custom LabVIEW program (NI, Austin Tex.). The impact magnitude was adjusted by setting the deflection of the impactor's internal spring and mechanical parameters for each impact were calculated (as described in Example 3).

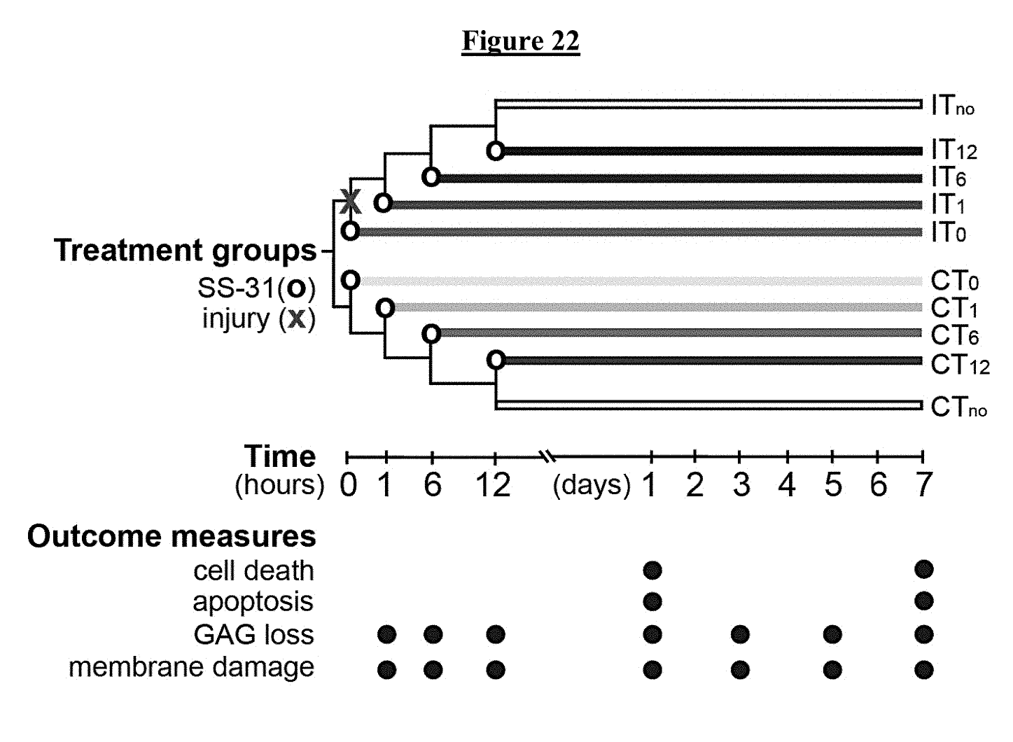

D-Arg-2′,6′-Dmt-Lys-Phe-NH₂ (SS-31) treatment. Following injury, explants were cut perpendicular to the articular surface into 2 hemicylinders using a custom cutting jig to ensure uniform geometry. Cartilage was kept moist at all times and each cut was performed with a fresh (unused) and lubricated cutting instrument. Handling of the experimental tissues was strictly minimized at each step. Explants were placed directly into an individual well of a 24-well untreated tissue culture plate containing a known volume (1.5 ml) of cartilage explant media. Cartilage hemicylinders were randomly assigned to one of 10 treatment groups (n=6/group, FIG. 22). Injured (I) and uninjured control (C) explants in the non-treated groups (ITₙₒ, CTₙₒ) were placed into wells containing only media. Explants in the time zero treatment groups (IT₀, CT₀) were placed directly into media containing 1 μM D-Arg-2′,6′-Dmt-Lys-Phe-NH₂(SS-31). Explants in the one-hour treatment groups (IT₁, CT₁) were placed into media only and D-Arg-2′,6′-Dmt-Lys-Phe-NH₂(1 μM) was added to the wells 1 hour after impact. Explants in the six-hour treatment groups (IT₆, CT₆) were placed into media only and D-Arg-2′,6′-Dmt-Lys-Phe-NH₂ (1 μM) was added 6 hours after impact. Explants in the twelve-hour treatment groups (IT₁₂, CT₁₂) were placed into media only and D-Arg-2′,6′-Dmt-Lys-Phe-NH₂ (1 μM) was added 12 hours after impact. Explants were maintained under standard tissue culture conditions (36° C. and 21% O₂) for 7 days. Medium was sampled at 1, 6, and 12 hours, and at 3, 5, and 7 days after impact, and stored at −80° C. until biochemical assays were performed. Culture media was replaced with fresh media (i.e., no D-Arg-2′,6′-Dmt-Lys-Phe-NH₂) after 24 hours, then every other day for the duration of the experiment. After imaging was complete on day 7, cartilage explants were lyophilized and weighed for normalization of LDH and DMMB data.

Chondrocyte viability. At 1 or 7 days, cartilage was rinsed three times, then placed in PBS containing calcein AM (2 μM) and ethidium homodimer (1 μM) for 30 minutes at 37° C. in the dark, to stain live and dead cells, respectively. Explants were then rinsed in PBS and imaged on a Leika SP5 confocal microscope. Digital z-stacked images were acquired in two channel sequential scans (green; 488/498-544 and red; 514/563-663 nm excitation/emission, respectively) using a modified 3D scanning protocol consisting of 10 z-stacked 512×512 pixel (387.5 μm×387.5 μm) images spaced 10 pm apart in the z plane at 10× magnification.

The number of live, dead, and total cells in each image was quantified using a custom ImageJ (Mac OS X version 10.2, Wayen Rasband, U.S. National Institutes of Health, Bethesda, Md.) macro. Pixel intensity threshold, max/min particle size, and particle circularity were optimized for each imaging channel based on manual counts of a minimum of 5 z-stacks obtained from control and impacted explants. Following optimization, all images were digitally analyzed using the same macro as follows: red and green channels for each image were thresholded based on mean pixel intensity of that image, then individual particles were identified and counted based on particle size and circularity. For each image, the % of dead cells was calculated as the number of dead cells counted in the red channel divided by the total number of cells (live+dead) counted in both channels. To minimize artifact from cell death occurring at the cut surface, the average % dead cell for all images in a z-stack was calculated, and any individual image with a value outside one standard deviation from the mean was excluded from analysis. This resulted in few images being excluded (mean 1 image per stack, range 0-2). The final reported values for live, dead and total cells were calculated as the mean of a minimum of 3 z-stacks obtained for each explant.

Apoptosis (activated caspase staining). At 1 or 7 days, cartilage was rinsed with PBS three times, then placed in PBS containing CellEvent Caspase 3/7 (Molecular Probes, Eugene, Oreg.) to stain for activated caspase activity. Explants were imaged in on a Leika SP5 confocal microscope. Digital z-stacked images were acquired in two channel sequential scans (488/498-544 excitation/emission, respectively to image apoptotic cells and reflectance to highlight collagen in the extracellular matrix) using a modified 3D scanning protocol consisting of 10 z-stacked 512×512 pixel (387.5 μm×387.5 μm) images spaced 10 μm apart in the z plane at 10× magnification. The number of caspase-positive cells per field were counted using a custom ImageJ macro, as described above and expressed as the number of apoptotic cells per mm².

Cell membrane damage. Imaging studies were validated with biochemical assays performed on cartilage conditioned media. As a measure of cell membrane damage, the release of lactate dehydrogenase (LDH) from cartilage explants into culture media was quantified using an LDH activity assay (Sigma-Aldrich) that detects NADH, which is reduced from NAD by LDH. The assay was executed per the manufacturer's instructions. Briefly, equal volumes of cartilage conditioned media and kit reagent were added to a 96-well assay plate (Corning, Corning, N.Y.) and absorbance was measured at 450 nm in 5-minute intervals by a spectrophotometric microplate reader (Tecan Safire; Männedorf, Switzerland). Using a standard curve generated by dilutions of NADH, LDH activity was calculated following subtraction of background media values and expressed as milliunits of LDH per ml of cartilage conditioned media.

Cartilage GAG loss. To determine if D-Arg-2′,6′-Dmt-Lys-Phe-NH₂ could provide structural protection after impact injury, the loss of glycosaminoglycan (GAG) was determined by routine 1,9-dimethyl-methylene blue dye binding (DMMB) assay. Briefly, media samples were digested using papain (Sigma Aldrich, St. Louis, Mo.) 0.25 mg/ml at 65° C. for 4 hours. A standard curve was prepared using chondroitin-4-sulfate (Sigma Aldrich). Equal volumes of sample and DMMB dye (Sigma Aldrich) were mixed in a 96-well plate. Total GAG content was read fluorometrically, and expressed as the total GAG released into cartilage conditioned media over the culture period per μg dry weight of cartilage.

Statistical analysis. Data was analyzed using a linear mixed effects model, with a random effect of trial and fixed effects of injury (I, C), treatment time (TX, T0, T1, T6), and response time, including all interactions. Comparisons between groups were performed using Tukey's HSD method. Residual analyses were performed to ensure the assumptions of normality and homogeneous variance were met. Differences were considered statistically significant when p≦0.05. All statistical analyses were performed using JMP Pro Version 11.0 (SAS Inc.) software.

### Results

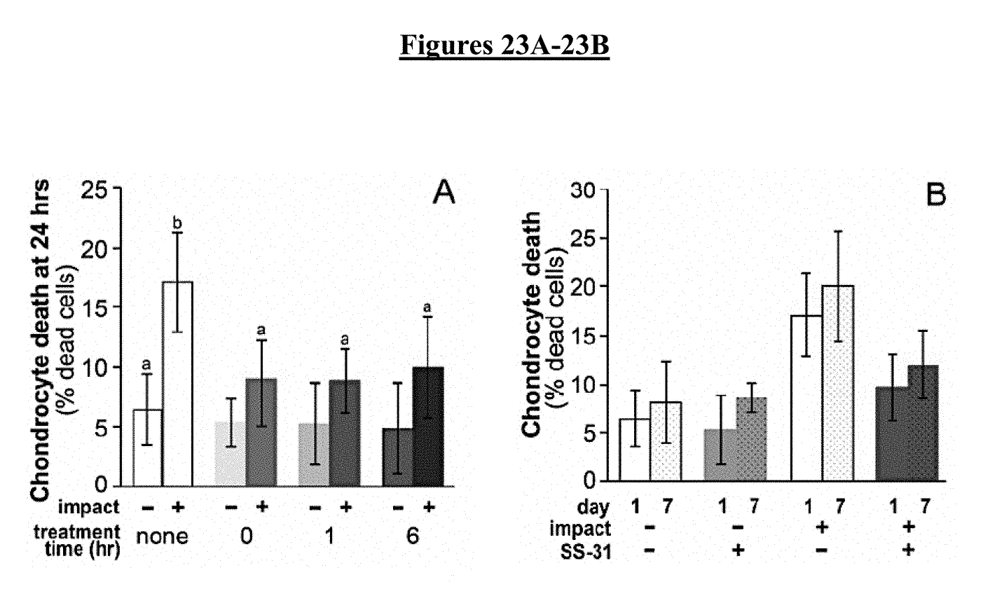

Chondrocyte death and apoptosis. Cartilage impact resulted in a roughly 3-fold increase in the amount of cell death at 1 day post-injury (FIG. 23A-C). At 1 day, D-Arg-2′,6′-Dmt-Lys-Phe-NH₂ potently reduced cell death in all injured treatment groups (IT₀; p=0.0007, IT₁; p<0.0001, IT₆; p=0.0003); this equates to over 50% reduction in impact-induced chondrocyte death in treated versus untreated explants (IT_{X}). In all injury+treatment groups, chondrocyte viability was similar to un-injured controls (CT_{X}; p=0.16). Cell death also did not differ between injury groups treated at 0, 1, or 6 hours, indicating no effect of treatment time (p=0.93). When chondrocyte viability was assessed on day 7, the same trends were present; D-Arg-2′,6′-Dmt-Lys-Phe-NH₂ prevented impact-induced cell death to a similar degree, regardless of whether treatment was applied immediately following, at 1 hour or at 6 hours after impact (FIG. 23A-C). Note that since no effect of treatment time was detected on day 1 or day 7, groups T₀, T₁ and T₆ were collapsed and represented as a single treatment group in FIGS. 23A-C. Cell death was not significantly different on day 1 and day 7 in uninjured, non-treated (CTX) explants (p=0.98), indicating baseline chondrocyte viability was maintained for the 7-day culture period (FIG. 23A-C).

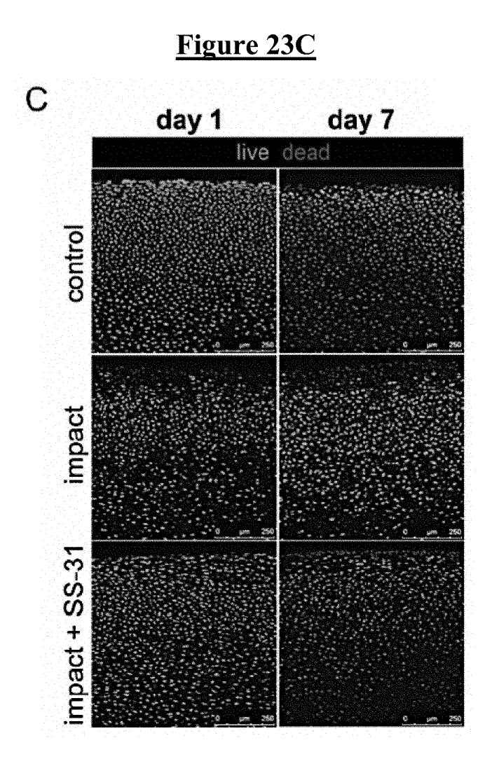

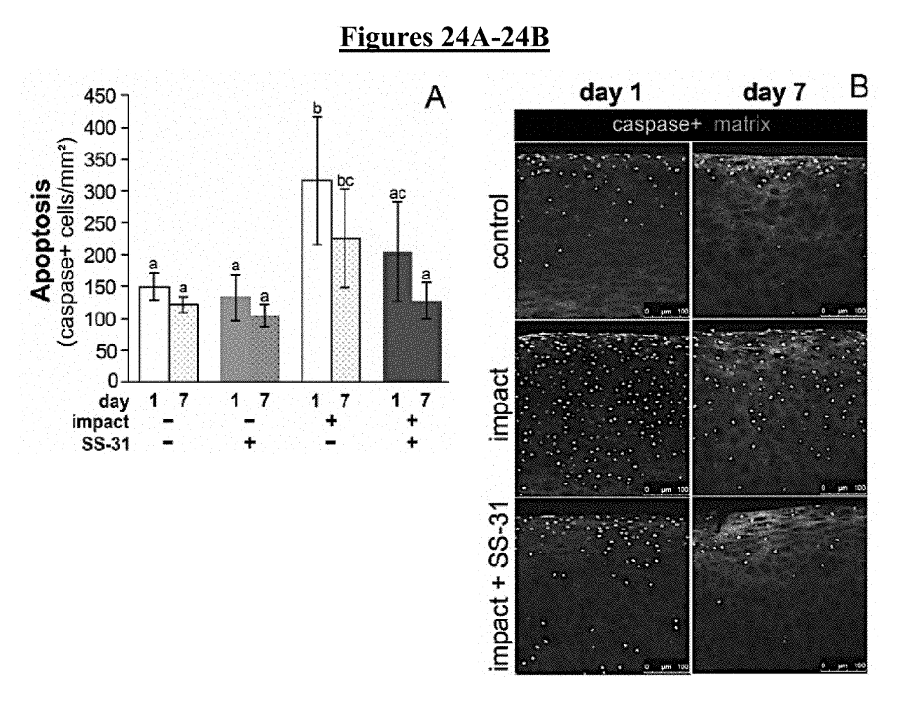

Activated caspase 3/7 staining of injured explants on day 1 and 7 revealed an increase in the number of apoptotic cells throughout the depth of the cartilage (FIG. 24A-B). D-Arg-2′,6′-Dmt-Lys-Phe-NH₂ prevented impact-induced apoptosis at 1 day (p=0.007) and 7 days (p=0.04) after cartilage injury. There was a trend toward fewer apoptotic cells on day 7 than day 1 in all groups, most notably in injured, treated explants but this difference did not reach statistical significance (p=0.07).

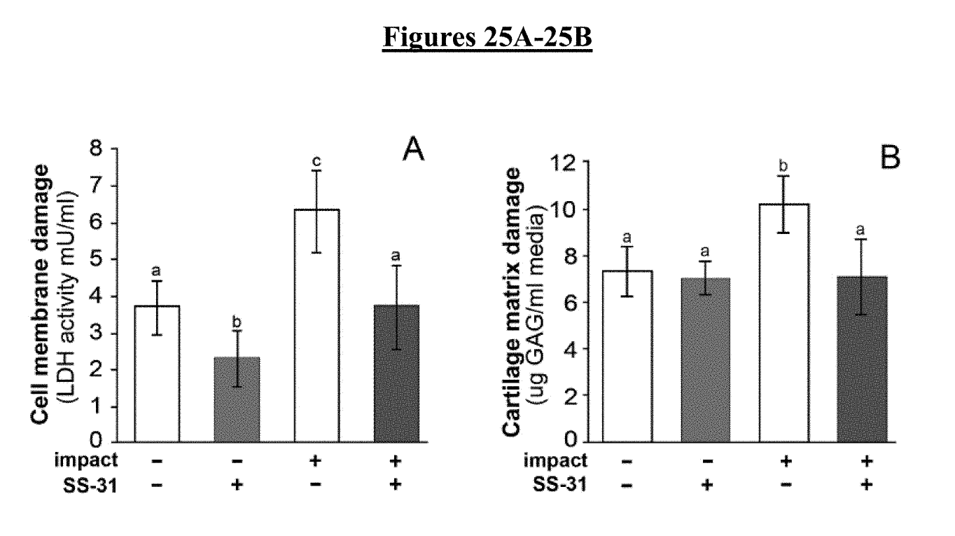

Cell membrane damage and cartilage GAG loss. Cumulative cell membrane damage, quantified by LDH activity in cartilage conditioned media over the 7 days following injury, was approximately twofold lower after injury in treated than untreated cartilage (p=0.0005, FIG. 25A). D-Arg-2′,6′-Dmt-Lys-Phe-NH₂ also appears to have a protective effect against cell membrane damage in uninjured controls; uninjured, treated samples had a lower cumulative LDH than untreated controls (p=0.05). Impact-induced GAG loss was decreased by ˜30% in explants treated with D-Arg-2′,6′-Dmt-Lys-Phe-NH₂ (p=0.002, FIG. 25B).

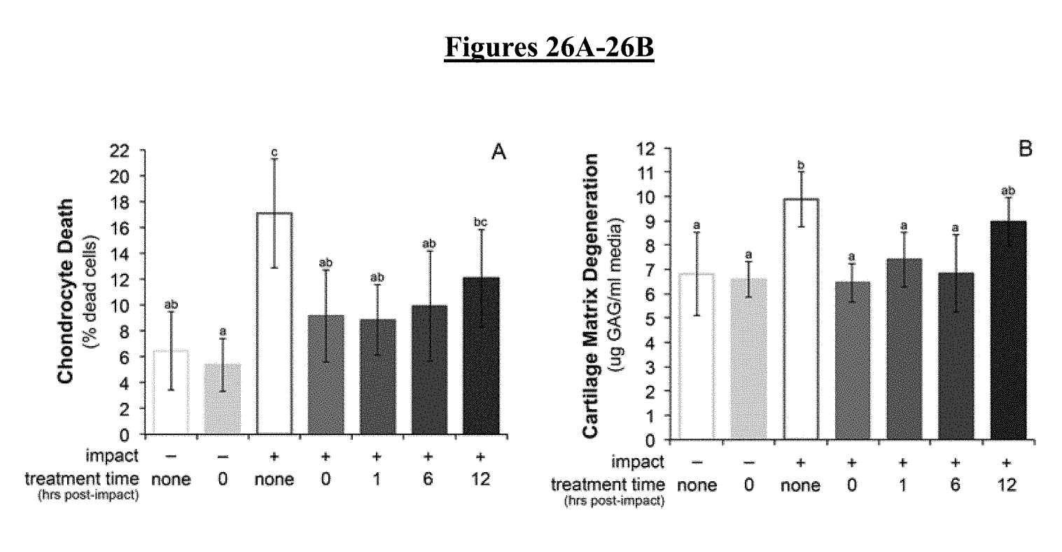

Chondrocyte death in explants treated with D-Arg-2′,6′-Dmt-Lys-Phe-NH₂ (SS-31) at 0, 1, 6, or 12 hours had reduced chondrocyte death as compared to untreated, injured controls (FIG. 26A).

Cartilage matrix degeneration, measured by glycosaminoglycan (GAG) loss into the media, in explants treated with D-Arg-2′,6′-Dmt-Lys-Phe-NH₂ (SS-31) at 0, 1, or 6 hours was equivalent to uninjured controls. Additionally, explants treated with D-Arg-2′,6′-Dmt-Lys-Phe-NH₂ at 12 hours had reduced cartilage matrix degeneration as compared to untreated, injured controls (FIG. 26B).

These results show that treatment with D-Arg-2′,6′-Dmt-Lys-Phe-NH₂ reduced mechanical injury-induced chondrocyte death, apoptosis, cell membrane damage, and cartilage matrix degradation. Additionally, the results show that D-Arg-2′,6′-Dmt-Lys-Phe-NH₂ reduced mechanical injury-induced chondrocyte death, apoptosis, cell membrane damage, and cartilage matrix degradation even after treatment with D-Arg-2′,6′-Dmt-Lys-Phe-NH₂ was delayed by up to 12 hours after injury. As such, D-Arg-2′,6′-Dmt-Lys-Phe-NH₂ is useful in preventing chondrocyte death, apoptosis, cell membrane damage, and matrix degradation. Accordingly, the aromatic-cationic peptides of the present technology, or a pharmaceutically acceptable salt thereof, such as acetate, tartrate salt, or trifluoroacetate salt, including but not limited to D-Arg-2′,6′-Dmt-Lys-Phe-NH₂, are useful in treating, preventing, or ameliorating OA or PTOA.

### Example 7

### Prevention of Chondrocyte Death and Cartilage Degeneration Following Mechanical Injury

### Methods

Cartilage are harvested from the knee joints of 4 neonatal bovids (n=30 explants). Cartilage explants are treated with 1 μM D-Arg-Dmt-Lys-Phe-NH₂ (SS-31) before mechanical injury. Cartilage explants are subjected to unconfined compression (24.0±1.4 MPa peak stress; 53.8±5.3 GPa/s peak stress rate) using a validated single-impact subcritical damage model. Control cartilage is not treated with D-Arg-Dmt-Lys-Phe-NH₂ before injury.

Cartilage is maintained in condition media after injury. Cartilage conditioned media (CCM) was sampled every 24 hours after injury (T₂₄₋₁₆₈) for 7 days.

Explants are stained with calcein AM and ethidium homodimer (for live and dead cells, respectively) to assess chondrocyte viability at T₂₄ and T₁₆₈ using confocal microscopy. Live, dead and total cell numbers are quantified in z-stacked digital images using a custom ImageJ macro. To quantify cell membrane damage, CCM is analyzed using a colorimetric lactate dehydrogenase (LDH) activity assay, and cumulative cell membrane damage over the 7-day incubation period is determined for each explant.

Cartilage matrix degradation is quantified by measuring GAG loss into the media via DMMB assay.

### Results

It is anticipated that treatment with D-Arg-2′,6′-Dmt-Lys-Phe-NH₂ before mechanical injury will reduce chondrocyte death, cell membrane damage, and cartilage matrix degradation in treated cartilage as compared to untreated controls.

The results will show that treatment with D-Arg-2′,6′-Dmt-Lys-Phe-NH₂ before mechanical injury prevents chondrocyte death, cell membrane damage, and cartilage matrix degradation after mechanical injury. Accordingly, the aromatic-cationic peptides of the present technology, or a pharmaceutically acceptable salt thereof, such as acetate, tartrate salt, or trifluoroacetate salt, including but not limited to D-Arg-2′,6′-Dmt-Lys-Phe-NH2, are useful in preventing OA or PTOA.

### Equivalents

The present technology is not to be limited in terms of the particular embodiments described in this application, which are intended as single illustrations of individual aspects of the present technology. Many modifications and variations of this present technology can be made without departing from its spirit and scope, as were apparent to those skilled in the art. Functionally equivalent methods and apparatuses within the scope of the present technology, in addition to those enumerated herein, were apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As were understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as were understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. A method for treating or preventing osteoarthritis (OA) in a subject in need thereof comprising administering an effective amount D-Arg-2′,6′-Dmt-Lys-Phe-NH₂, or a pharmaceutically acceptable salt thereof.

2. The method of claim 1, wherein the osteoarthritis is post-traumatic osteoarthritis (PTOA).

3. The method of claim 1, wherein the osteoarthritis is caused by mechanical injury.

4. The method of claim 1, wherein the osteoarthritis is located in the shoulder, hand, foot, ankle, toe, hip, spine, jaw, or knee.

5. The method of claim 1, wherein the aromatic-cationic peptide, or pharmaceutically acceptable salt thereof, is administered orally, topically, intranasally, intraperitoneally, intravenously, subcutaneously, intraarticularly, or transdermally.

6. The method of claim 3, wherein the peptide is administered within about 1 to 12 hours following mechanical injury.

7. The method of claim 1, wherein treatment or prevention comprises reducing or ameliorating one or more symptoms of osteoarthritis is selected from the group consisting of joint pain; joint swelling; joint clicking; joint cracking and/or creaking; joint stiffness; limited range of motion in a joint; pain in the groin, buttocks, inside knee, or thigh; grating or scraping sensation during movement of a knee; pain or tenderness in a toe joint; and swelling in ankles or toes.

8. A method for treating or preventing post-traumatic osteoarthritis (PTOA) in a subject in need thereof comprising administering an effective amount D-Arg-2′,6′-Dmt-Lys-Phe-NH₂, or a pharmaceutically acceptable salt thereof.

9. The method of claim 8, wherein the PTOA is caused by mechanical injury.

10. The method of claim 8, wherein the PTOA is located in the shoulder, hand, foot, ankle, toe, hip, spine, jaw, or knee.

11. The method of claim 8, wherein the D-Arg-2′,6′-Dmt-Lys-Phe-NH₂, or pharmaceutically acceptable salt thereof, is administered orally, topically, intranasally, intraperitoneally, intravenously, subcutaneously, intraarticularly, or transdermally.

12. The method of claim 8, wherein the peptide is administered within about 1 to 12 hours following mechanical injury.

13. The method of claim 8, wherein treatment or prevention comprises reducing or ameliorating one or more symptoms of osteoarthritis is selected from the group consisting of joint pain; joint swelling; joint clicking; joint cracking and/or creaking; joint stiffness; limited range of motion in a joint; pain in the groin, buttocks, inside knee, or thigh; grating or scraping sensation during movement of a knee; pain or tenderness in a toe joint; and swelling in ankles or toes.

14. A method for reducing cartilage degeneration and/or chondrocyte death after mechanical injury in a subject in need thereof comprising administering D-Arg-2′,6′-Dmt-Lys-Phe-NH₂, or a pharmaceutically acceptable salt thereof.

15. The method of claim 14, wherein the cartilage degeneration and/or chondrocyte death is associated with osteoarthritis (OA) or post-traumatic osteoarthritis (PTOA).

16. The method of claim 14, wherein the cartilage degeneration and/or chondrocyte death is caused by mechanical injury.

17. The method of claim 14, wherein the cartilage degeneration and/or chondrocyte death is located in the shoulder, hand, foot, ankle, toe, hip, spine, jaw, or knee.

18. The method of claim 14, wherein the aromatic-cationic peptide, or pharmaceutically acceptable salt thereof, is administered orally, topically, intranasally, intraperitoneally, intravenously, subcutaneously, intraarticularly, or transdermally.

19. The method of claim 14, wherein the peptide is administered within about 1 to 12 hours following mechanical injury.

20. The method of claim 14, wherein reducing cartilage degeneration and/or chondrocyte death reduces or ameliorates one or more symptoms of osteoarthritis is selected from the group consisting of joint pain; joint swelling; joint clicking; joint cracking and/or creaking; joint stiffness; limited range of motion in a joint; pain in the groin, buttocks, inside knee, or thigh; grating or scraping sensation during movement of a knee; pain or tenderness in a toe joint; and swelling in ankles or toes.
